(19) United States
(12) Patent Application Publication
(10) Pub. No.: US 2014/0050735 A1
(43) Pub. Date: Feb. 20, 2014

(54) TREATMENT OF FISTULIZING CHROHN'S DISEASE

(71) Applicants: Anne Ruehl, Basel (CH); Gerhard Rogler, Zurich (CH); Michael Scharl, Zurich (CH)

(72) Inventors: Anne Ruehl, Basel (CH); Gerhard Rogler, Zurich (CH); Michael Scharl, Zurich (CH)

(21) Appl. No.: 13/985,732

(22) PCT Filed: Feb. 15, 2012
(86) PCT No.: PCT/IB12/50699

§ 371 Date: Oct. 30, 2013

Related U.S. Application Data
(60) Provisional application No. 61/443,829, filed on Feb. 17, 2011.

(51) Int. Cl. C07K 16/24; A61K 45/06; A61K 39/395

(52) U.S. Cl. CPC C07K 16/244; A61K 39/3955; A61K 45/06
USPC 424/139.1; 530/387.9

(57) ABSTRACT

This invention is in the field of Crohn's disease. In particular, it relates to the treatment of fistulas in Crohn's disease using anti-IL-13 antibodies. The antibody may be an IgG and in particular may be the anti-IL-13 antibody 01951/G12.

### TECHNICAL FIELD

This invention is in the field of Crohn's disease. In particular, it relates to the treatment of fistulas in Crohn's disease using anti-IL-13 antibodies. The antibody may be an IgG and in particular may be the anti-IL13 antibody 01951/G12.

### BACKGROUND

Crohn's disease (CD) is a chronic, relapsing/remitting inflammatory disease of the gastrointestinal (GI) tract. The regions of the GI tract most often affected by CD are the small intestine and the colon, including the ano-rectum. The inflammation and ulcerations of CD can extend throughout all layers of the intestinal wall in both the small and large intestines. Common symptoms of CD include diarrhea, abdominal pain, rectal bleeding, and weight loss as well as complications such as intestinal abscesses, fistulas, and intestinal obstructions. CD can become clinically manifest in many different ways including fibrostenotic (stricturing), or nonperforating, nonstricturing (inflammatory), or predominantly perforating (fistulizing) disease. Patients with fistulizing CD tend to have a more aggressive disease course. Fistulas can be either external (enterocutaneous or perianal) or internal, such as entero-enteral or entero-cystic. The cumulative incidence of CD fistulas is 33% and 50% 10 and 20 yrs, respectively, after diagnosis.

Morbidity is greatly increased in patients with fistulizing disease resulting in considerable negative impact on patients' quality of life. Perianal fistulas can lead to fecal incontinence, abscess formation and anal strictures; they may be further associated with pain, abscesses, and drainage. The treatment of fistulas depends on many factors, including location, severity, and previous surgical history.

Overall, CD fistulas are difficult to treat, rarely heal spontaneously and frequently require surgery. Before the introduction of biologic agents, most fistulas required surgical intervention, and the rate of fistula recurrence was estimated to be 30-40% (1-3). The current standard of care is antibiotics (metronidazole/ciprofloxacin—1st line), immunosuppressives (6-MP/azathioprine—2nd line) and biologics (anti-TNFα's—3rd line, or ‘top-down’ 1st line). Calcineurin inhibitors are being tested. Of note, some of the standard-of-care therapies for fistulizing Crohn's disease (e.g. azathioprine and 6-MP) are teratogenic.

The advent of biologics has expanded therapeutic treatment options and changed the practitioners' goal of treatment for fistulas from reduction in fistula drainage to true closure of the fistula tract. However, approximately 50% of patients do not respond to anti-TNFα's and hence, given the risk of incontinence associated with aggressive surgical procedures, there remains an unmet medical need for new and improved therapies for fistula treatment in Crohn's disease.

### SUMMARY OF THE INVENTION

It has been discovered that IL-13 mediated signaling may contribute to fistula formation in patients suffering from Crohn's disease. The invention therefore provides an anti-IL-13 antibody which inhibits or neutralizes the activity of IL-13 for use in the treatment or prevention of fistulas in patients suffering from Crohn's disease. The use of an anti-IL-13 antibody which inhibits or neutralizes the activity of IL-13 in the manufacture of a medicament for the treatment or prevention of fistulas in patients suffering from Crohn's disease is also provided as well as a method of treating or preventing formation of fistulas in patients suffering from Crohn's disease comprising administering an anti-IL-13 antibody which inhibits or neutralizes the activity of IL-13 to a subject in need thereof.
Preferably the antibody of the invention comprises one or more of the CDRs selected from the list consisting of: (a) the V_{H} CDR1s shown in SEQ ID NOs: 1, 2, 6 or 7 (b) the V_{H} CDR2s shown in SEQ ID NOs: 3 or 8, (c) the V_{H} CDR3s shown in SEQ ID NOs: 4, 5, 9 or 10, (d) the V_{L} CDR1s shown in SEQ ID NOs: 11, 16, 17 or 18, (e) the V_{L} CDR2s shown in SEQ ID NOs: 12 or 19, (f) the V_{L} CDR3s shown in SEQ ID NOs: 13, 14, 15, 20, 21 or 22.
Preferably the antibody of the invention comprises a heavy chain variable region CDR1 of SEQ ID NO: 7; a heavy chain variable region CDR2 of SEQ ID NO: 8; a heavy chain variable region CDR3 of SEQ ID NO: 10; a light chain variable region CDR1 of SEQ ID NO: 17; a light chain variable region CDR2 of SEQ ID NO: 19; and a light chain variable region CDR3 of SEQ ID NO: 21.
The antibody provided preferably comprises a heavy chain variable region as recited in SEQ ID NO: 31 and a light chain variable region as recited in SEQ ID NO: 33, more preferably the antibody comprises a heavy chain as recited in SEQ ID NO: 41 and a light chain as recited in SEQ ID NO: 39.
The antibody provided preferably binds to IL-13 with a K_{D} of 1×10⁻⁹ M or less. The antibody provided is preferably formulated with a pharmaceutically acceptable carrier.
In a preferred embodiment of the invention the antibody, use or method provided, the antibody is co-administered sequentially or simultaneously with an anti inflammatory therapeutic agent.
The invention further provides a kit comprising a first component and a second component wherein the first component is an anti-IL-13 antibody or pharmaceutical composition comprising an anti-IL-13 antibody and the second component is instructions.
The kit provided may further comprise a third component comprising one or more of the following: syringe or other delivery device, adjuvant, or pharmaceutically acceptable formulating solution.

### LIST OF FIGURES

FIG. 1. TGFβ induced the secretion of IL-13 from fistula colonic lamina propria fibroblasts (CLPF). CLPF derived from patients with non-fistulising (n=2) or fistulising disease (n=3) were treated with TGFβ (50 ng/ml). Histograms show mRNA levels of (A) SNAIL1 or (B) SLUG relative to the respective untreated controls. (C) Non-fistula (n=3) and fistula (n=5) CLPF were treated with TGFβ. Histogram demonstrates IL-13 concentration in cell supernatants in pg/ml. (D) Fistula CLPF were treated with TGFβ (50 ng/ml) or TNF (100 ng/ml) and protein levels of PTPN2 and the loading control, β-Actin, are demonstrated by representative Western blots (n=3). Histogram represents IL-13 concentration in the cell supernatant of the cells shown in the Western blot above. (E) T₈₄ cells were transfected with either non-specific or PTPN2-specific siRNA constructs and treated with TNF (100 ng/ml). Histogram demonstrates IL-13 concentration in cell supernatants in percentage of respective controls (n=4). (F) Protein levels of PTPN2 and the loading control, β-Actin in HT29 IEC treated with TGFβ (50 g/ml) are demonstrated by representative Western blots (n=3). Asterisks denote significant differences from untreated non-fistula CLPF (C) or the respective control (E) (*=p<0.05). #=p<0.05 vs. 24 h TNF treatment of control siRNA cells.

FIG. 2. IL-13 and IL-13Rα₁ protein in fistula specimens from CD patients. Surgically resected fistulas were immunohistochemically stained (A) IL-13 was clearly visible in cells lining the fistula tracts (grey arrows) as well as in IEC of deformed crypts adjacent to the fistulas. IEC of crypts with normal appearance feature almost no IL-13 staining (white arrows). (B) IL-13Rα₁ reveals a similar staining pattern as IL-13. It was clearly detectable in cells lining the fistula tracts as well as in IEC of deformed crypts adjacent to the fistulas. For each staining, images from two different fistulas from two separate patients are shown and are representative for all of the investigated fistulas (n=7). Pictures in the right column represent the enlargements (magnification 40-fold) of the pictures in the left column (magnification: 5-fold).

FIG. 3. IL-13 induces mRNA levels of SLUG and b6-integrin in HT29 IEC. HT29 cells were treated with IL-13 (100 ng/ml) for 30 min or 24 h, respectively. Histograms represent the mRNA levels of (A) SLUG (n=3), (B) β6-integrin (n=3), (C) SNAIL1 (n=3), (D) TGFβ (n=3) and (E) PTPN2 relative to the respective controls. (F) HT29 cells were transfected with either non-specific or SLUG-specific siRNA constructs and treated with IL-13 (100 ng/ml) for 24 h. Histogram shows the mRNA level of β6-integrin relative to the respective controls. Asterisks indicate significant differences compared to the respective control (*=p<0.05, **=p<0.01, ***=p<0.001). #=p<0.05 vs. 24 h treatment of control siRNA cells.

FIG. 4. IL-13 induces phosphorylation of STAT6 and ERK1/2 as well as expression of claudin-2 in HT29 cells. Cells were treated with IL-13 (100 ng/ml) for 30 min or 24 h, respectively. (A) Representative Western blots demonstrate levels of phosphorylated (Tyr⁶⁴¹) and total STAT6 as well as of phosphorylated (Thr²⁰²/Tyr²⁰⁴) and total ERK1/2 (n=3). (B) Representative Western blots show protein levels of β-catenin and the loading control, β-actin. The histogram represents the densitometric analysis of three similar experiments. (C) Protein levels of claudin-2 and the loading control. β-actin, are demonstrated by representative Western blots. Densitometric analysis of three similar experiments is represented by the histogram below. (D) Representative Western blots show protein levels of E-cadherin, occludin and the loading control, β-actin (n=3). Asterisks indicate significant differences vs. the respective control (**=p<0.01).

FIG. 5. Chronic administration of TGFβ, but not of IL-13, induced EMT in a HT29 IEC spheroid model. HT29 cells were seeded as “hanging drops” for 7 d. Then cells were either left untreated, treated with TGFβ (20 ng/ml) or treated with IL-13 (100 ng/ml) for further 7 d. Untreated spheroids do not show any alteration in cell formation. TGFβ treated spheroids almost completely disassemble after 7 d, indicative for the onset of EMT in these cells. In contrast, IL-13 treatment does not cause an obvious cell disassembling, suggesting that IL-13 does not induce EMT in this cell model. Each image is representative for three similar experiments per condition. Pictures in column 2 and 4 represent the enlargement (magnification: 40-fold) of columns 1 and 3 (magnification: 20-fold).

FIG. 6. TGFβ induces mRNA levels of IL-13 and SNAIL1 in HT29 spheroids. HT29 spheroids were treated with TGFβ (20 ng/ml) for up to 7 days. Histograms represent the mRNA levels of (A) IL-13, (B) SNAIL1, (C) β6-integrin and (D) SLUG relative to the respective controls (n=3 each). Asterisks indicate significant differences compared to the respective controls (*=p<0.05, **=p<0.01).

FIG. 7. IL-13 induces mRNA levels of SLUG and β6-integrin in HT29 spheroids. HT29 spheroids were treated with TGFβ (20 ng/ml) for up to 7 days. Histograms represent the mRNA levels of (A) SLUG, (B) β6-integrin and (C) SNAIL1 (n=3 each). (D) Representative Western blots show protein levels of E-cadherin, claudin-2 and the loading control, β-actin, in spheroids either left untreated or treated with IL-13 (100 ng/ml) or TGFβ (20 ng/ml). n=3 each. Asterisks indicate significant differences compared to the respective controls (*=p<0.05, ***=p<0.001).

FIG. 8. Basal levels of SLUG, β6-integrin and MMP-13 are elevated in fistula CLPF. Histograms show mRNA levels of (A) SLUG and (8) β6-integrin in CLPF derived from patient with non-fistulising (n=3) or fistulising (n=3) CD, respectively. These CLPF were then treated with IL-13 (100 ng/ml) for 30 min or 24 h, respectively. (C) Representative Western blots show levels of phosphorylated (Tyr⁵⁴¹) and total STAT6 as well as of phosphorylated (Thr²⁰²/Tyr²⁰⁴) and total ERK1/2 in CLPF derived from patient with non-fistulising (n=3) or fistulising (n=3) CD, respectively. (D) Representative Western blots show protein levels of full length and cleaved (activated) MMP-13, as well as of the loading control, β-actin, in CLPF derived from patient with non-fistulising (n=3) or fistulising (n=3) CD, respectively.

### DESCRIPTION OF THE INVENTION

Pathophysiologically, the transmural inflammation characteristic of CD predisposes patients to the formation of fistulas, and impaired wound healing appears to be involved. While no preclinical animal models are available to further explore this indication, fistulizing CD likely involves dysregulated tissue remodeling processes that occur in the context of chronic transmural inflammation: Tissue repair and consecutive fibrosis result from excessive extracellular matrix (ECM) synthesis due to enhanced myofibroblast activity in conjunction with reduced activity of proteolytic/ECM degrading enzymes, while on the other hand inflammation-induced ulcer formation, i.e. tissue destruction, is driven by oxygen metabolites, activated immune cells, and up-regulated ECM degrading enzymes like matrix metalloproteinases (MMPs) and serine-proteases.

Based on the current understanding of the role of IL-13 in the context of CD (35), IL-13 may have anti-inflammatory properties. Therefore, an anti IL-13 treatment could lead to an exacerbation of the inflammatory activity in CD patients. Anti IL-13 therapy in CD is therefore not normally considered an option.

It has recently been demonstrated that epithelial-to-mesenchymal transition (EMT) related events are present in and around CD-associated fistulas (6). EMT represents the transformation from a differentiated, polarized epithelial cell to a mesenchymal-like cell featuring a myofibroblast phenotype. As a specific characteristic these EMT cells downregulate their intercellular connections and display both, epithelial markers, such as E-cadherin or cytokeratines 8 and 20, as well as mesenchymal markers, such as vimentin or α-SMA (7-8). On a functional level, EMT is essential for embryogenesis, organ development and wound repair, but is also associated with tissue fibrosis as well as with tumour growth and metastasis (7-9).
About two thirds of the CD-associated fistulas are non-epithelialized fistulas and are covered by myofibroblast-like “transitional cells” (TC) (6). In or around the tracts of CD-associated fistulas, we detected nuclear localisation of the transcription factors SNAIL1 and SLUG, indicative for their activation, as well as elevated levels of β6-integrin, TGFβ and TNF. In contrast, we observed a decreased protein expression of epithelial markers, such as the cell adhesion molecule. E-cadherin (6). TGFβ is well known as a key mediator of EMT, induces SNAIL1 expression and can, similar to TNF, induce EMT in vitro (10-14). Of notice, β6-integrin and SLUG have been positively correlated with the invasive potential of tumour cells and the extent of EMT (15-17).
The cytokine IL-13 is mainly secreted by immune cells, especially Th₂ cells (18). It can bind to two different receptors, IL-13 receptor alpha 1 (IL-13Rα₁) and IL-13Rα₂, whereby IL-13Rα₁ is mainly regarded as the signal-transducing receptor and IL-13Rα₂ as a decoy receptor (19). IL-13 has been implicated in the pathogenesis of diseases featuring a hyper-responsive immune system, such as airway hyper-responsiveness, allergic inflammation or mastocytosis (18). Interestingly, IL-13 has also been implicated in the pathogenesis of tissue fibrosis in organ systems, such as the lung or the liver (20-21). In this setting, IL-13 causes the production of proline, which is important for collagen synthesis, or may act directly on fibroblasts triggering pro-fibrotic effects (18). A further fibrosis-inducing pathway of IL-13 involves the secretion and activation of TGFβ indicating that the growth factor could be a downstream mediator of the cytokine (22). In contrast, the data about the role for IL-13 in tumour growth and invasion are conflicting, since IL-13 has recently been associated with increased invasiveness and metastasis of ovarian and pancreatic cancers (23-26), but can obviously also inhibit tumour growth, for example the growth of breast or renal cell cancer (27-28).

Here, we demonstrate that TGFβ induces SNAIL1 as well as IL-13 mRNA expression in primary human colonic lamina propria fibroblasts (CLPF) derived from CD patients. High levels of IL-13 and IL-13Rα₁ were detected in TC lining the tracts of CD-associated fistulas. In an intestinal epithelial cell (IEC) model of EMT, IL-13 induced SLUG and β6-integrin levels, whereas chronic TGFβ administration resulted in concomitant elevation of SNAIL1 and IL-13 mRNA expression. However, the mediators exerted their effects with opposing kinetics. Our data show that IL-13 is present in CD-associated fistulas and induces the expression of genes associated with invasive cell growth suggesting an important role for the cytokine in the pathogenesis of such fistulas.

We have interpreted these findings to indicate that IL-13 is involved in driving the tissue remodeling that accompanies fistula formation in CD, so that anti IL-13 therapy will be a useful treatment for this patient group.

The IL-13 polypeptide has the below sequence. The N-terminal 34 amino acid residues (in italics) is a signal peptide. The mature cytokine thus has 112 amino acid residues. Anti-IL-13 antibodies will bind to an epitope on the mature polypeptide.

Interleukin 13 amino acid sequence:
1 MHPLLNPLLL ALGLMALLLT TVIALTCLGG FASPGPVPPS TALRELIEEL VNITQNQKAP
61 LCNGSMVWSI NLTAGMYCAA LESLINVSGC SAIEKTQRML SGFCPHKVSA GQFSSLHVRD
TKIEVAQFVK DLLLHLKKLF REGRFN (SEQ ID No. 43)

### Antibodies Used in the Invention

In principle any anti-IL-13 antibody which inhibits or neutralizes the activity of IL-13 may be used in the invention. Such antibodies are known in the art, see for example in WO2005/007699, U.S. Pat. No. 6,468,528, WO03007685, WO03034984, US20030143199, US2004028650, US20040242841, US2004023337, US20040248260, US20050054055, US20050065327, WO2006/124451, WO2006/003407, WO2005/062967, WO2006/085938, WO2006/055638, WO2007/036745, WO2007/080174 or WO2007/085815.

In a preferred embodiment, the antibody is 01951/G12 (SEQ ID No. 31 and 33), further described in WO2007/045477.

In one embodiment, the antibodies used in the invention have affinities to IL-13 in the low pM range and inhibit IL-13 induced signalling with an IC50 of about 10 nM. By low pM range we mean 100 pM or less, preferably 500 pM or less, preferably 10 pM or less, more preferably 1 pM or less.
The inhibition or neutralization of the activity of IL-13 can be assessed by measuring the inhibition of inflammatory mediator release, as for example using the eotaxin release from human lung fibroblasts as disclosed in WO2007/045477. In preferred embodiments anti IL-13 antibodies of the invention inhibits IL-13 induced eotaxin release from human lung fibroblasts with an IC₅₀ less than 10 nM, 5 nM, 2.5 nM, 1.0 nM, 0.5 nM, or less.
In a preferred embodiment the antibodies used in the invention comprise one or more of the following CDRs. The CDRs listed in table 3a and 4a were determined according to the Kabat definition (E. Kabat et al, 1991, Sequences of Proteins of immunological interest, 5^{th} edition, public health Service, HIH, Bethesda, Md.

TABLE 3

| | | SEQ ID | | SEQ ID | | SEQ ID |
|---|---|---|---|---|---|---|
| | | No. | | No. | | No. |
| Antibody | HCDR1 | HCDR1 | HCDR2 | HCDR2 | HCDR3 | HCDR3 |
| 01471/G6 | GFTESNYG | 1 | IWYDGSN | 3 | VKGSGDIP | 4 |
| 03161/H2 | GFTFSNYG | 1 | IWYDGSN | 3 | VKGSGDIP | 4 |
| 01951/G12 | GFTESSYG | 2 | IWYDGSN | 3 | ARLWFGDLD | 5 |
| 01771/E10 | GFTFSSYG | 2 | IWYDGSN | 3 | ARLWFGDLD | 5 |

TABLE 3a

| | | SEQ ID | | SEQ ID | | SEQ ID |
|---|---|---|---|---|---|---|
| | | No. | | No. | | No. |
| Antibody | HCDR1 | HCDR1 | HCDR2 | HCDR2 | HCDR3 | HCDR3 |
| 01471/G6 | NYGMH | 6 | IIWYDGSNKYYADSVKG | 8 | GSGDIPFDY | 9 |
| 03161/H2 | NYGMH | 6 | IIWYDGSNKYYADSVKG | 8 | GSGDIPFDY | 9 |
| 01951/G12 | SYGMH | 7 | IIWYDGSNKYYADSVKG | 8 | LWFGDLDAFDI | 10 |
| 01771/E10 | SYGMH | 7 | IIWYDGSNKYYADSVKG | 8 | LWFGDLDAFDI | 10 |

TABLE 4

| | | SEQ | | SEQ | | SEQ |
|---|---|---|---|---|---|---|
| | | ID | | ID | | ID |
| | | No. | | No. | | No. |
| Antibody | LCDR1 | LCDR1 | LCDR2 | LCDR2 | LCDR3 | LCDR3 |
| 01471/G6 | QSVSSY | 11 | DA | 12 | HQRSHWPPI | 13 |
| 03161/H2 | QSVSSY | 11 | DA | 12 | HQRSHWPPI | 13 |
| 01951/G12 | QSVSSY | 11 | DA | 12 | QQRSSWPPV | 14 |
| 01771/E10 | QSVSSY | 11 | DA | 12 | HQRSSWPPI | 15 |

TABLE 4a

| | | SEQ ID | | SEQ ID | | SEQ ID |
|---|---|---|---|---|---|---|
| | | No. | | No. | | No. |
| Antibody | LCDR1 | LCDR1 | LCDR2 | LCDR2 | LCDR3 | LCDR3 |
| 01471/G6 | RASOSVSSYLA | 16 | DASNRAT | 19 | HQRSHWPPIFT | 20 |
| 03161/H2 | RASQSVSSYLA | 16 | DASNRAT | 19 | HQRSHWPPIFT | 20 |
| 01951/G12 | RAGQSVSSYLV | 17 | DASNRAT | 19 | QQRSSWPPVYT | 21 |
| 01771/E10 | RASOSVSSYLA | 18 | DASNRAT | 19 | HQRSSWPPIFT | 22 |

The sequences of the antibodies of the previous tables, including framework regions, are shown below. The full IgG1 antibody light and heavy chain constant regions are also shown below, incorporating, as an example, the variable regions of antibody 01951/G12 (emboldened).

### 01471/G6 Antibody Sequence

### (i) HC Variable Region

The HC variable amino acid sequence for 01471/G6 is shown in SEQ ID NO: 23 and is encoded by the nucleotide sequence shown in SEQ ID NO: 24

| E V Q L V E S G G G V V Q P G R S L R L | |
|---|---|
| gaagtgcagctggtggagtctgggggaggcgtggtccagcctgggaggtccctgagactc | 60 |
| S C A A S G F T F S N Y G M H W V R Q A | |
| tcctgtgcagcgtctggattcaccttcagtaactatggcatgcactgggtccgccaggct | 120 |
| P G K G L E W V A I I W Y D G S N K Y Y | |
| ccaggcaaggggctggagtgggtggcaattatatggtatgatggaagtaataaatactat | 180 |
| A D S V K G R F T I S R D N S K N T L Y | |
| gcagactccgtgaagggccgattcaccatctccagagacaattccaagaacacgctgtat | 240 |
| L Q M N S L R A E D T A V Y Y C V K G S | |
| ctgcaaatgaacagtctgagagccgaggacacggctgtgtattactgtgtgaaaggatct | 300 |
| G D I P F D Y W G Q G T L V T (SEQ ID NO: 23) | |
| ggggatattccctttgactactggggccagggaaccctggtcacc (SEQ ID NO: 24) | 345 |

### (ii) LC Variable Region

The LC variable amino acid sequence for 01471/G6 is shown in SEQ ID NO: 25 and is encoded by the nucleotide sequence shown in SEQ ID NO: 26

| E I V L T Q S P A T L S S S P G E R A T | |
|---|---|
| gaaattgtgttgacgcagtctccagccaccctgtcttcgtctccaggggaaagagccacc | 60 |
| L S C R A S Q S V S S Y L A W Y Q Q K P | |
| ctctcctgcagggccagtcagagtgttagcagctacttagcctggtaccaacagaaacct | 120 |
| G Q A P R L L I Y D A S N R A T G I P A | |
| ggccaggctcccaggctcctcatctatgatgcatccaacagggccactggcatcccagcc | 180 |
| R F S G S G S G T D F T L T I S S L E P | |
| aggttcagtggcagtgggtctgggacagacttcactctcaccatcagcagcctagagcct | 240 |
| E D F A V Y Y C H Q R S H W P P I F T F | |
| gaagattttgcagtctattactgtcatcagcgtagccactggcctcccatattcactttc | 300 |
| G P G T (SEQ ID NO: 25) | |
| ggccctgggacc (SEQ ID NO: 26) | 312 |

### 03161/H2 Antibody

### (i) HC Variable Region

The HC variable amino acid sequence for 03161/H2 is shown in SEQ ID NO: 27 and is encoded by the nucleotide sequence shown in SEQ ID NO: SEQ ID No. 28

| E V Q L V E S G G G V V Q P G R S L R L | |
|---|---|
| gaagtgcagctggtggagtctgggggaggcgtggtccagcctgggaggtccctgagactc | 60 |
| S C A A S G F T F S N Y G M H W V R Q A | |
| tcctgtgcagcgtctggattcaccttcagtaactatggcatgcactgggtccgccaggct | 120 |
| P G K G L E W V Q I I W Y D G S N K Y Y | |
| ccaggcaaggggctggagtgggtggcaattatatggtatgatggaagtaataaatactat | 180 |
| A D S V K G R F T I S R D N S K N T L Y | |
| gcagactccgtgaagggccgattcaccatctccagagacaattccaagaacacgctgtat | 240 |
| L Q M N S L R A E D T A V Y Y C V K G S | |
| ctgcaaatgaacagtctgagagccgaggacacggctgtgtattactgtgtgaaaggatct | 300 |
| G D I P F D Y W G Q G T L V T (SEQ ID NO: 27) | |
| ggggatattccctttgactactggggccagggaaccctggtcacc (SEQ ID NO: 28) | 345 |

### (ii) IC Variable Region

The LC variable amino acid sequence for 03161/H2 is shown in SEQ ID NO: 29 and is encoded by the nucleotide sequence shown in SEQ ID NO: 30

| E I V L T Q S P A T L S S S P G E R A T | |
|---|---|
| gaaattgtgttgacgcagtccccagccaccctgtcttcgtctccaggggaaagagccacc | 60 |
| L S C R A S Q S V S S Y L A W Y Q Q K P | |
| ctctcctgcagggccagtcagagtgttagcagctacttagcctggtaccaacagaaacct | 120 |
| G Q A P R L L I Y D A S N R A T G T P A | |
| ggccaggctcccaggctcctcatctatgatgcatccaacagggccactggcaccccagcc | 180 |
| R F S G S G S G T D F T L T I S S L E P | |
| aggttcagtggcagtgggtctgggacagacttcactctcaccatcagcagcctagagcct | 240 |
| E D F A V Y Y C H Q R S H W P P I F T F | |
| gaagattttgcagtctattactgtcatcagcgtagccactggcctcccatattcactttc | 300 |
| G P G T (SEQ ID NO: 29) | |
| ggccctgggacc (SEQ ID NO: 30) | 312 |

### 01951/G12 Antibody Sequence

### (i) HC Variable Region

The HC variable amino acid sequence for 01951/G12 is shown in SEQ ID NO: 31 and is encoded by the nucleotide sequence shown in SEQ ID NO: 32

| E V Q L V E S G G G V V Q P G R S L R L | |
|---|---|
| gaagtgcagctggtggagtctgggggaggcgtggtccagcctgggaggtccctgagactc | 60 |
| S C A A S G F T F S S Y G M H W V R Q A | |
| tcctgtgcagcgtctggattcaccttcagtagctatggcatgcactgggtccgccaggct | 120 |
| P G K G L E W V A I I W Y D G S N K Y Y | |
| ccaggcaaggggctggagtgggtggcaattatatggtatgatggaagtaataaatactat | 180 |
| A D S V K G R F T I S R D N S K N T L Y | |
| gcggactccgtgaagggccgattcaccatctccagagacaattccaagaacacgctgtat | 240 |
| L Q M N S L R A E D T A V Y Y C A R L W | |
| ctgcaaatgaacagcctgagagccgaggacacggctgtgtattactgtgcgaggctatgg | 300 |
| F G D L D A F D I W G Q G T M V T (SEQ ID NO: 31) | |
| ttcggggacttagatgcttttgatatctggggccaagggacaatggtcacc (SEQ ID NO: 32) | 351 |

### (ii) LC Variable Region

The LC variable amino acid sequence for 01951/G12 is shown in SEQ ID NO: 33 and is encoded by the nucleotide sequence shown in SEQ ID NO: 34

| E I V L T Q S P A T L S L S P G E R A I | |
|---|---|
| gaaattgtgttgacgcagtctccagccaccctgtctttgtctccaggggaaagagccatc | 60 |
| L S C R A G Q S V S S Y L V W Y Q Q K P | |
| ctctcctgcagggccggtcagagtgttagcagttacttagtctggtaccaacagaaacct | 120 |
| G Q A P R L L I Y D A S N R A T G I P A | |
| ggccaggctcccaggctcctcatctatgatgcatccaacagggccactggcatcccagcc | 180 |
| R F S G S G S G T D F T L T I S S L E P | |
| aggttcagtggcagtgggtctgggacagacttcactctcaccatcagcagcctagagcct | 240 |
| E D F A V Y Y C Q Q R S S W P P V Y T F | |
| gaagattttgcagtttattactgtcagcagcgcagcagctggcctccggtgtacactttt | 300 |
| G Q G T (SEQ ID NO: 33) | |
| ggccaggggacc (SEQ ID NO: 34) | 312 |

### 01771/E10 Antibody Sequence

### (i) HC Variable Region

The HC variable amino acid sequence for 01771/E10 is shown in SEQ ID NO: 35 and is encoded by the nucleotide sequence shown in SEQ ID NO: 36

| Q V Q L V Q S G G G V V Q P G R S L R L | |
|---|---|
| caggtgcagctggtgcagtctgggggaggcgtggtccagcctgggaggtccctgagactc | 60 |
| S C A A S G F T F S S Y G M H W V R Q A | |
| tcctgtgcggcgtctggattcaccttcagtagctatggcatgcactgggtccgccaggct | 120 |
| P G K G L E W V A I I W Y D G S N K Y Y | |
| ccaggcaaggggctggagtgggtggcaattatatggtatgatggaagtaataaatactat | 180 |
| A D S V K G R F T I S R D N S K N T L Y | |
| gcggactccgtgaagggccgattcaccatctccagagacaattccaagaacacgctatat | 240 |
| L Q M N S L R A E D T A V Y Y C A R L W | |
| ctacaaatgaacagcctgagagccgaggacacggctgtgtattactgtgcgaggctatgg | 300 |
| F G D L D A F D I W G Q G T M V T (SEQ ID NO: 35) | |
| ttcggggacttagatgcttttgatatctggggccaagggacaatggtcacc (SEQ ID NO: 36) | 351 |

### (ii) LC Variable Region

The LC variable amino acid sequence for 01771/E10 is shown in SEQ ID NO: 37 and is encoded by the nucleotide sequence shown in SEQ ID NO: 38

| E I V L T Q S P A T L S L S P G E R A T | |
|---|---|
| gaaattgtgttgacgcagtctccagccaccctgtctttgtctccaggggaaagagccacc | 60 |
| L S C R A S Q S V S S Y L A W Y Q Q K P | |
| ctctcctgcagggccagtcagagtgttagcagctacttagcctggtaccaacagaaacct | 120 |
| G Q A P R L L I Y D A S N R A T G I P A | |
| ggccaggctcccaggctcctcatctatgatgcatccaacagggccactggcatcccagcc | 180 |
| R F S G S G S G T D F T L T I S S L E P | |
| aggttcagtggcagtgggtctgggacagacttcactctcaccatcagcagcctagagcct | 240 |
| E D F A V Y Y C H Q R S S W P P I F T F | |
| gaagattttgcggtttattactgtcatcagcgtagcagctggcccccgatattcactttc | 300 |
| G P G T (SEQ ID NO: 37) | |
| ggccctgggacc (SEQ ID NO: 38) | 312 |

### Full Antibody IgG1 Light Chain Sequence Incorporating the Variable Region of Antibody 01951/G12 (Emboldened)

The LC amino acid sequence is shown in SEQ ID NO: 39 and is encoded by the nucleotide sequence of SEQ ID NO: 40

| | M S V L T Q V L A L L L L W L T G |
|---|---|
| 1 | ATGAGTGTGC TCACTCAGGT CCTGGCGTGG CTGCTGCTGT GGCTTACAGG |
| | T R C E I V L T Q S P A T L S L S |
| 51 | TACGCGTTGT GAAATTGTGT TGACGCAGTC TCCAGCCACC CTGTCTTTGT |
| | P G E R A I L S C R A G Q S V S |
| 101 | CTCCAGGGGA AAGAGCCATC CTCTCCTGCA GGGCCGGTCA GAGTGTTAGC |
| | S Y L V W Y Q Q K P G Q A P R L L |
| 151 | AGTTACTTAG TCTGGTACCA ACAGAAACCT GGCCAGGCTC CCAGGCTCCT |
| | I Y D A S N R A T G I P A R F S G |
| 201 | CATCTATGAT GCATCCAACA GGGCCACTGG CATCCCAGCC AGGTTCAGTG |
| | S G S G T D F T L T I S S L E P |
| 251 | GCAGTGGGTC TGGGACAGAC TTCACTCTCA CCATCAGCAG CCTAGAGCCT |
| | E D F A V Y Y C Q Q R S S W P P V |
| 301 | GAAGATTTTG CAGTTTATTA CTGTCAGCAG CGCAGCAGCT GGCCTCCGGT |
| | Y T F G Q G T K L E I K R T V A A |
| 351 | GTACACTTTT GGCCAGGGGA CCAAGCTTGA AATCAAACGA ACTGTGGCTG |
| | P S V F I F P P S D E Q L K S G |
| 401 | CACCATCTGT CTTCATCTTC CCGCCATCTG ATGAGCAGTT GAAATCTGGA |
| | T A S V V C L L N N F Y P R E A K |
| 451 | ACTGCCTCTG TTGTGTGCCT GCTGAATAAC TTCTATCCCA GAGAGGCCAA |
| | V Q W K V D N A L Q S G N S Q E S |
| 501 | AGTACAGTGG AAGGTGGATA ACGCCCTCCA ATCGGGTAAC TCCCAGGAGA |
| | V T E Q D S K D S T Y S L S S T |
| 551 | GTGTCACAGA GCAGGACAGC AAGGACAGCA CCTACAGCCT CAGCAGCACC |
| | L T L S K A D Y E K H K V Y A C E |
| 601 | CTGACGCTGA GCAAAGCAGA CTACGAGAAA CACAAAGTCT ACGCCTGCGA |
| | V T H Q G L S S P V T K S F N R G |
| 651 | AGTCACCCAT CAGGGCCTGA GCTCGCCCGT CACAAAGAGC TTCAACAGGG |
| | E C * (SEQ ID NO: 39) |
| 701 | GAGAGTGTTA G (SEQ ID NO: 40) |

### Full Antibody IgG1 Heavy Chain Sequence Incorporating the Variable Region of Antibody 01951/G12 (Emboldened)

The HC amino acid sequence is shown in SEQ ID NO: 41 and is encoded by the nucleotide sequence of SEQ ID NO: 42

| | M A W V W T L P F L M A A A Q S V |
|---|---|
| 1 | ATGGCTTGGG TGTGGACCTT GCCATTCCTG ATGGCAGCTG CCCAAAGTGT |
| | Q A E V Q L V E S G G G V V Q P G |
| 51 | CCAGGCAGAA GTGCAGCTGG TGGAGTCTGG GGGAGGCGTG GTCCAGCCTG |
| | R S L R L S C A A S G F T F S S |
| 101 | GGAGGTCCCT GAGACTCTCC TGTGCAGCGT CTGGATTCAC CTTCAGTAGC |
| | Y G M H W V R Q A P G K G L E W V |
| 151 | TATGGCATGC ACTGGGTCCG CCAGGCTCCA GGCAAGGGGC TGGAGTGGGT |
| | A I I W Y D G S N K Y Y A D S V K |
| 201 | GGCAATTATA TGGTATGATG GAAGTAATAA ATACTATGCG GACTCCGTGA |
| | G R F T I S R D N S K N T L Y L |
| 251 | AGGGCCGATT CACCATCTCC AGAGACAATT CCAAGAACAC GCTGTATCTG |
| | Q M N S L R A E D T A V Y Y C A R |
| 301 | CAAATGAACA GCCTGAGAGC CGAGGACACG GCTGTGTATT ACTGTGCGAG |
| | L W F G D L D A F D I W G Q G T M |
| 351 | GCTATGGTTC GGGGACTTAG ATGCTTTTGA TATCTGGGGC CAAGGGACAA |
| | V T V S S A S T K G P S V F P L |
| 401 | TGGTCACCGT CTCCTCAGCC TCCACCAAGG GCCCATCGGT CTTCCCCCTG |
| | A P S S K S T S G G T A A L G C L |
| 451 | GCACCCTCCT CCAAGAGCAC CTCTGGGGGC ACAGCGGCCC TGGGCTGCCT |
| | V K D Y F P E P V T V S W N S G A |
| 501 | GGTCAAGGAC TACTTCCCCG AACCGGTGAC GGTGTCGTGG AACTCAGGCG |
| | L T S G V H T F P A V L Q S S G |
| 551 | CCCTGACCAG CGGCGTGCAC ACCTTCCCGG CTGTCCTACA GTCCTCAGGA |
| | L Y S L S S V V T V P S S S L G T |
| 601 | CTCTACTCCC TCAGCAGCGT CGTGACCGTG CCCTCCAGCA GCTTGGGCAC |
| | Q T Y I C N V N H K P S N T K V D |
| 651 | CCAGACCTAC ATCTGCAACG TGAATCACAA GCCCAGCAAC ACCAAGGTGG |
| | K R V E P K S C D K T H T C P P |
| 701 | ACAAGAGAGT TGAGCCCAAA TCTTGTGACA AAACTCACAC ATGCCCACCG |
| | C P A P E L L G G P S V F L F P P |
| 751 | TGCCCAGCAC CTGAACTCCT GGGGGGACCG TCAGTCTTCC TCTTCCCCCC |
| | K P K D T L M I S R T P E V T C V |
| 801 | AAAACCCAAG GACACCCTCA TGATCTCCCG GACCCCTGAG GTCACATGCG |
| | V V D V S H E D P E V K F N W Y |
| 851 | TGGTGGTGGA CGTGAGCCAC GAAGACCCTG AGGTCAAGTT CAACTGGTAC |
| | V D G V E V H N A K T K P R E E Q |
| 901 | GTGGACGGCG TGGAGGTGCA TAATGCCAAG ACAAAGCCGC GGGAGGAGCA |
| | Y N S T Y R V V S V L T V L H Q D |
| 951 | GTACAACAGC ACGTACCGTG TGGTCAGCGT CCTCACCGTC CTGCACCAGG |
| | W L N G K E Y K C K V S N K A L |
| 1001 | ACTGGCTGAA TGGCAAGGAG TACAAGTGCA AGGTCTCCAA CAAAGCCCTC |
| | P A P I E K T I S K A K G Q P R E |
| 1051 | CCAGCCCCCA TCGAGAAAAC CATCTCCAAA GCCAAAGGGC AGCCCCGAGA |
| | P Q V Y T L P P S R E E M T K N Q |
| 1101 | ACCACAGGTG TACACCCTGC CCCCATCCCG GGAGGAGATG ACCAAGAACC |
| | V S L T C L V K G F Y P S D I A |
| 1151 | AGGTCAGCCT GACCTGCCTG GTCAAAGGCT TCTATCCCAG CGACATCGCC |
| | V E W E S N G Q P E N N Y K T T P |
| 1201 | GTGGAGTGGG AGAGCAATGG GCAGCCGGAG AACAACTACA AGACCACGCC |
| | P V L D S D G S F F L Y S K L T V |
| 1251 | TCCCGTGCTG GACTCCGACG GCTCCTTCTT CCTCTATAGC AAGCTCACCG |
| | D K S R W Q Q G N V F S C S V M |
| 1301 | TGGACAAGAG CAGGTGGCAG CAGGGGAACG TCTTCTCATG CTCCGTGATG |
| | H E A L H N H Y T Q K S L S L S P |
| 1351 | CATGAGGCTC TGCACAACCA CTACACGCAG AAGAGCCTCT CCCTGTCCCC |
| | G K * (SEQ ID NO: 41) |
| 1401 | GGGTAAATGA (SEQ ID NO: 42) |

It will be readily apparent to the ordinarily skilled artisan that novel VH and VL sequences can be created by substituting one or more VH and/or VL CDR region sequences with structurally similar sequences from the CDR sequences shown herein for monoclonal antibodies useful in the present invention.

As used herein, the term “antibody” means a polypeptide comprising a framework region from an immunoglobulin gene or fragments thereof that specifically binds and recognizes an epitope, e.g. an epitope found on IL-13, as described above. Thus, the term antibody includes whole antibodies (such as monoclonal, chimeric, humanised and human antibodies), including single-chain whole antibodies, and antigen-binding fragments thereof. The term “antibody” includes antigen-binding antibody fragments, including single-chain antibodies, which can comprise the variable regions alone, or in combination, with all or part of the following polypeptide elements: hinge region, CH₁, CH₂, and CH₃ domains of an antibody molecule. Also included within the definition are any combinations of variable regions and hinge region, CH₁, CH₂, and CH₃ domains. Antibody fragments include, e.g., but are not limited to, Fab, Fab′ and F(ab′)₂, Fd, single-chain Fvs (scFv), single-chain antibodies, disulphide-linked Fvs (sdFv) and fragments comprising either a V_{L} or V_{H} domain. Examples include: (i) a Fab fragment, a monovalent fragment consisting of the V_{L}, V_{H}, C_{L} and CH₁ domains; (ii) a F(ab′)₂ fragment, a bivalent fragment comprising two Fab fragments linked by a disulphide bridge at the hinge region; (iii) a Fd fragment consisting of the V_{H} and CH₁ domains; (iv) a Fv fragment consisting of the V_{L} and V_{H} domains of a single arm of an antibody, (v) a dAb fragment (Ward et al., Nature 341: 544-546, 1989; Muyldermans et al., TIBS 24; 230-235, 2001), which consists of a V_{H} domain; and (vi) an isolated complementarity determining region (CDR). The term “antibody” includes single domain antibodies, maxibodies, minibodies, intrabodies, diabodies, triabodies, tetrabodies, v-NAR and bis-scFv (see, e.g., Hollinger & Hudson, Nature Biotechnology, 23, 9, 1126-1136 (2005)). Antigen binding portions of antibodies can be grafted into scaffolds based on polypeptides such as Fibronectin type III (Fn3) (see U.S. Pat. No. 6,703,199, which describes fibronectin polypeptide monobodies). Antigen binding portions can be incorporated into single chain molecules comprising a pair of tandem Fv segments (VH-CH1-VH-CH1) which, together with complementary light chain polypeptides, form a pair of antigen binding regions (Zapata et al., Protein Eng. 8(10):1057-1062 (1995); and U.S. Pat. No. 5,641,870).

Preferably, the antibodies used in the invention bind specifically to IL-13. Preferably, the antibodies used in the invention do not cross-react with an antigen other than IL-13.

As used herein, an antibody that “specifically binds to IL-13” is intended to refer to an antibody that binds to IL-13 with a K_{D} of 1×10⁻⁸ M or less, 1×10⁻⁹ M or less, or 1×10⁻¹⁰ M or less. An antibody that “cross-reacts with an antigen other than IL-13” is intended to refer to an antibody that binds that antigen with a K_{D} of 0.5×10⁻⁸ M or less, 5×10⁻⁹ M or less, or 2×10⁻⁹ M or less.

In an alternative embodiment, the antibody used in the invention is one which cross-blocks one or more of the antibodies recited above. By “cross-blocks” we mean an antibody which interferes with the binding of another antibody to IL-13. Such interference can be detected, for example, using a competition assay using Biacore or ELISA. Such competition assays are described in WO2008/133722.

### Methods for Monitoring Treatment

Included within the scope of the invention are methods of monitoring whether treatment of fistulas in CD patients using an anti-IL-13 antibody have been successful. The simplest method, although not the most accurate, will be whether the subject being treated has noticed an amelioration of symptoms.

Other methods include detecting the expression of various biomarkers such as TGF-β, periostin, eotaxin-1, procollagen type I C-terminal propeptide (PICP) and the N-terminal pro-peptide of collagen type III (PIIINP). IL-4, as well as the degree of phosphorylation of STAT6.

Such methods comprise assessing the level of expression of a chosen biomarker in a subject being treated and comparing said level of expression to a control level (such as the level of expression in the subject prior to treatment or the level in an untreated subject), wherein a level that is different to said control level is indicative of the treated subject responding to treatment.

The method may comprise the steps of:

a) measuring the expression of a biomarker indicative of fistula formation in a patient prior to treatment;
b) treating the patient with an anti-IL-13 antibody;
c) measuring the expression of said biomarker in the patient following treatment;
d) detecting a change in biomarker expression following treatment compared to the pre-treatment expression level, wherein said change in expression indicates a response to anti-IL-13 antibody treatment.

The measuring steps (a) and (c) above may be carried out on tissue samples obtained from the patient. The tissue sample being analysed may be blood, urine, saliva or other tissue from a tissue biopsy.

As an alternative, step (d) may comprise comparing the biomarker expression before and after treatment with control biomarker expression levels, wherein deviation from those control levels indicates a response to treatment with an anti-IL-13 antibody. Such control levels may be from a CD-free patient, a patient treated with placebo, or a patient treated with conventional anti-fistula medication.

Examples of biomarkers that may be measured include, but are not limited to TGF-β, periostin, eotaxin-1, PICP and PIIINP, IL-4, as well as the degree of phosphorylation of STAT6.

### Pharmaceutical Compositions

The antibodies used in the invention are generally formulated as a composition, e.g., a pharmaceutical composition, containing one or a combination of monoclonal antibodies, formulated together with a pharmaceutically acceptable carrier. For example, a pharmaceutical composition used in the invention can comprise a combination of antibodies that bind to different epitopes of IL-13 or that have complementary activities.

Pharmaceutical compositions used in the invention also can be administered in combination therapy, i.e., combined with other agents. For example, the combination therapy can include an anti-IL-13 antibody combined with an anti inflammatory agent. Such combinations may be administered simultaneously or sequentially. If administered sequentially, the period between administration of each agent may be a week or less, (e.g. a day or less, 12 hours or less, 6 hours or less, 1 hour or less, 30 minutes or less). The compositions are preferably formulated at physiological pH.

As used herein, “pharmaceutically acceptable carrier” includes any and all solvents, dispersion media, coatings, antibacterial and antifungal agents, isotonic and absorption delaying agents, and the like that are physiologically compatible. The carrier should be suitable for intravenous, intramuscular, subcutaneous, parenteral, spinal or epidermal administration (e.g., by injection or infusion). Depending on the route of administration, the active compound, i.e., antibody, immunoconjugate, or bispecific molecule, may be coated in a material to protect the compound from the action of acids and other natural conditions that may inactivate the compound.

Such pharmaceutical compositions may also include a pharmaceutically acceptable anti-oxidant. Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as, aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, one can include isotonic agents, for example, sugars, polyalcohols such as mannitol, sorbitol, or sodium chloride in the composition. Prolonged absorption of the injectable compositions can be brought about by including in the composition an agent that delays absorption for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, from about 0.1 percent to about 70 per cent, or from about 1 percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of sensitivity in individuals.

For administration of the antibody, the dosage ranges from about 0.0001 to about 100 mg/kg, and more usually about 0.01 to about 5 mg/kg, of the host body weight. For example dosages can be about 0.3 mg/kg body weight, about 1 mg/kg body weight, about 3 mg/kg body weight, about 5 mg/kg body weight, about 10 mg/kg body weight, about 20 mg/kg body weight, about 30 mg/kg body weight or within the range of about 1- about 30 mg/kg or about 1- about 10 mg/kg. An exemplary treatment regime entails administration about once per week, about once every two weeks, about once every three weeks, about once every four weeks, about once a month, about once every 3 months, about once every three to 6 months, about once every six months or about once a year. Dosage regimens for an anti-IL-13 antibody of the invention include about 1 mg/kg body weight or about 3 mg/kg body weight by intravenous administration, with the antibody being given using one of the following dosing schedules: about every four weeks for six dosages, then about every three months; about every three weeks; about 3 mg/kg body weight once followed by about 1 mg/kg body weight every three weeks.

In some methods, two or more monoclonal antibodies with different binding specificities are administered simultaneously or sequentially, in which case the dosage of each antibody administered falls within the ranges indicated. The combination could be an anti-IL-13 antibody combined with an anti-IL4 antibody. Antibody is usually administered on multiple occasions. Intervals between single dosages can be, for example, weekly, monthly, every three months, every six months or yearly. Intervals can also be irregular as indicated by measuring blood levels of antibody to the target antigen in the patient. In some methods, dosage is adjusted to achieve a plasma antibody concentration of about 1- about 1000 μg/ml and in some methods about 25- about 300 μg/ml.

Alternatively, antibody can be administered as a sustained release formulation, in which case less frequent administration is required. Dosage and frequency vary depending on the half-life of the antibody in the patient. In general, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated or until the patient shows partial or complete amelioration of symptoms of disease. Thereafter, the patient can be administered a prophylactic regime.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” of an anti-IL-13 antibody of the invention can result in a decrease in severity of disease symptoms, an increase in frequency and duration of disease symptom-free periods, or a prevention of impairment or disability due to the disease affliction.

Compositions used in the present invention can be administered by one or more routes of administration using one or more of a variety of methods known in the art. As will be appreciated by the skilled artisan, the route and/or mode of administration will vary depending upon the desired results. Routes of administration for antibodies of the invention include intravenous, intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Intravenous and subcutaneous administration are particularly preferred.

Alternatively, an antibody used in the invention can be administered by a nonparenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Therapeutic compositions can be administered with medical devices known in the art. For example, in one embodiment, the compositions can be administered with a needleless hypodermic injection device, such as the devices shown in U.S. Pat. Nos. 5,399,163; 5,383,851; 5,312,335; 5,064,413; 4,941,880; 4,790,824 or 4,596,556. Examples of well known implants and modules useful in the present invention include: U.S. Pat. No. 4,487,603, which shows an implantable micro-infusion pump for dispensing medication at a controlled rate; U.S. Pat. No. 4,486,194, which shows a therapeutic device for administering medicants through the skin; U.S. Pat. No. 4,447,233, which shows a medication infusion pump for delivering medication at a precise infusion rate, U.S. Pat. No. 4,447,224, which shows a variable flow implantable infusion apparatus for continuous drug delivery; U.S. Pat. No. 4,439,196, which shows an osmotic drug delivery system having multi-chamber compartments; and U.S. Pat. No. 4,475,196, which shows an osmotic drug delivery system. These patents are incorporated herein by reference. Many other such implants, delivery systems, and modules are known to those skilled in the art.

The invention also provides a kit comprising a first component and a second component wherein the first component is an anti-IL-13 antibody or pharmaceutical composition as described above and the second component is instructions. In one embodiment, said instructions teach of the use of the antibody for treating fistulizing CD. The kit may further include a third component comprising one or more of the following: syringe or other delivery device, adjuvant, or pharmaceutically acceptable formulating solution.

### General

The term “comprising” means “including” as well as “consisting” e.g. a composition “comprising” X may consist exclusively of X or may include something additional e.g. X+Y.

The term “about” in relation to a numerical value x means, for example, x±10%. References to a percentage sequence identity between two amino acid sequences means that, when aligned, that percentage of amino acids are the same in comparing the two sequences. This alignment and the percent homology or sequence identity can be determined using software programs known in the art, for example those described in section 7.7.18 of Current Protocols in Molecular Biology (F. M. Ausubel et al., eds., 1987) Supplement 30. A preferred alignment is determined by the Smith-Waterman homology search algorithm using an affine gap search with a gap open penalty of 12 and a gap extension penalty of 2. BLOSUM matrix of 62. The Smith-Waterman homology search algorithm is disclosed in Smith & Waterman (1981) Adv. Appl. Math. 2: 482-489

### EXAMPLES

### Example 1

### TGFβ Induces IL-13 Secretion from CD Fistula CLPF

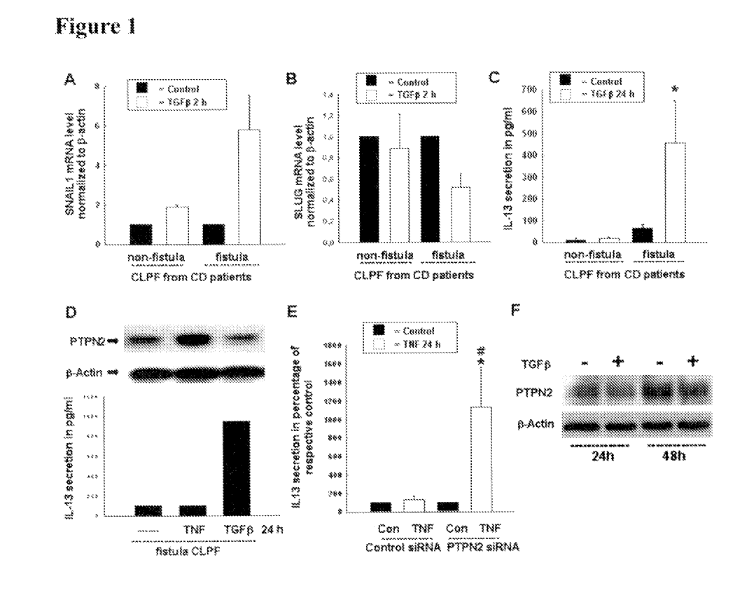

It has recently been shown that the EMT-associated transcription factor SNAIL1 is strongly detectable in TC cells alongside the fistula tracts of CD-associated fistulae (Scharl, in press). Therefore, we first studied whether TGFβ could induce SNAIL1 levels in CLPF derived from CD patients. In CLPF from patients with non-fistulising disease, treatment with TGFβ induced a two-fold increase in SNAIL1 mRNA expression and this effect was potentiated in CLPF from patients with fistulising disease (about 6-fold increase; FIG. 1A). In contrast, SLUG mRNA levels were not affected by TGFβ treatment in either fistulising or non-fistulising CLPF (FIG. 1B). In non-fistula CLPF, TGFβ was not sufficient to induce the secretion of IL-13. However, fistula CLPF featured a clearly elevated basal secretion of IL-13 (66.2+/−14.6 pg/ml vs. 11+/−10 pg/ml) and addition of TGFβ for 24 h resulted in a strong and significant increase in IL-13 secretion in the cell supernatant (455+/−193.6 pg/ml) (FIG. 1C). We then tested, whether TNF that plays an important role for CD pathogenesis, would also be able to induce IL-13 secretion from fistula CLPF. However, treatment of fistula CLPF with TNF was not sufficient to elevate IL-13 secretion, but induced protein expression of protein tyrosine phosphatase non-receptor type 2 (PTPN2). In contrast, TGFβ which was able to induce IL-13 secretion did the induce PTPN2 protein levels (FIG. 1D). To demonstrate a direct correlation between PTPN2 levels and the ability of cytokines or growth factors to stimulate IL-13 secretion, we knocked-down PTPN2 in T₈₄ IEC by siRNA constructs. While TNF treatment was not sufficient to induce IL-13 secretion in PTPN2-competent cells, it caused a strong secretion of IL-13 into cell supernatants of PTPN2-deficient cells (FIG. 1E). Similar to our findings in fistula fibroblasts treatment of HT29-IEC with TGFβ resulted in a slightly reduced, but still maintained PTPN2 protein level (FIG. 1F) that was obviously sufficient to prevent TGFβ-induced IL-13 secretion from these cells (data not shown). These data demonstrate that TGFβ induces the secretion of IL-13 from fistula CLPF and the extent of IL-13 secretion correlates with PTPN2 protein levels.

### Example 2

### IL-13 and IL-13 Receptor α₁ (IL-13Rα₁) are Strongly Detectable in TC Cells

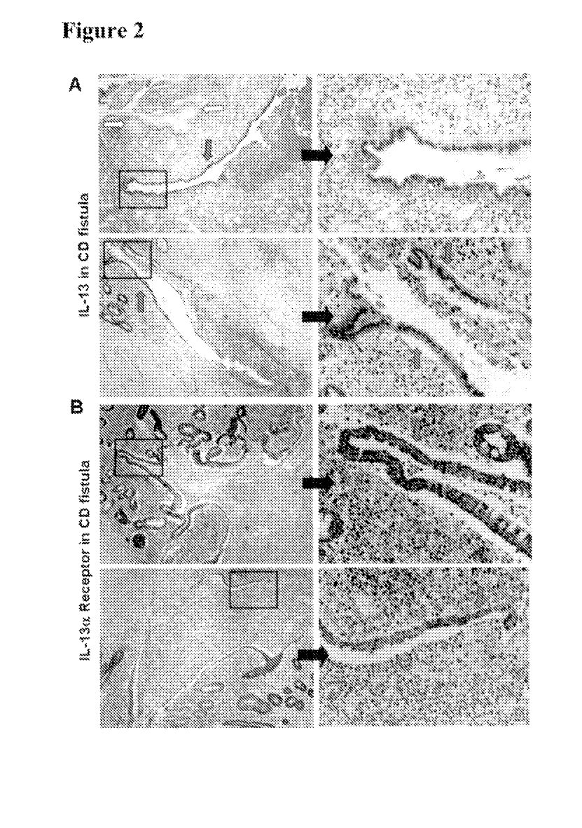

Since we have demonstrated that TGFβ induces IL-13 secretion from fistula CLPF we analyzed tissue specimen from fistula resectates derived from CD patients. By immunohistochemistry we observed a strong staining for IL-13 in TC covering the fistula tracts (FIG. 2A). Additionally, we found IL-13 staining in epithelial cells of deformed crypts directly next to the fistula tracts as well as in inflammatory infiltrates adjacent to the fistula tracts, whereas IEC of regular colonic crypts presented almost no IL-13 staining (FIG. 2A). A similar staining pattern could be observed for IL-13Rα₁. The cytokine receptor showed a strong staining in TC of CD fistulae as well as in epithelial-like cells lining crypt-like structures adjacent to the fistulae (FIG. 2B). These findings demonstrate that IL-13 and IL-13Rα₁ are expressed in TC cells and epithelial cells of deformed crypts alongside and next to CD-associated fistulae and suggest an involvement of IL-13 in the pathogenesis of CD fistulae.

### Example 3

### IL-13 Induces SLUG and β6-Integrin Expression in HT29-IEC

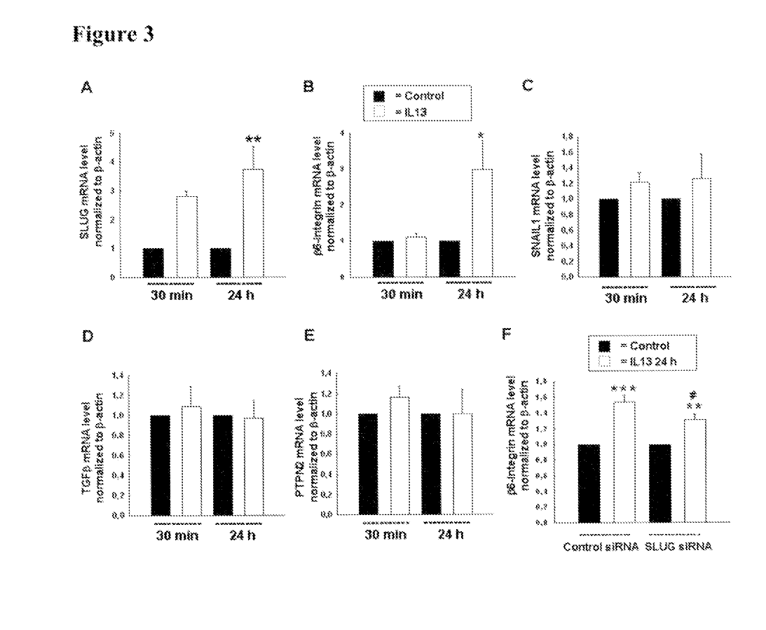

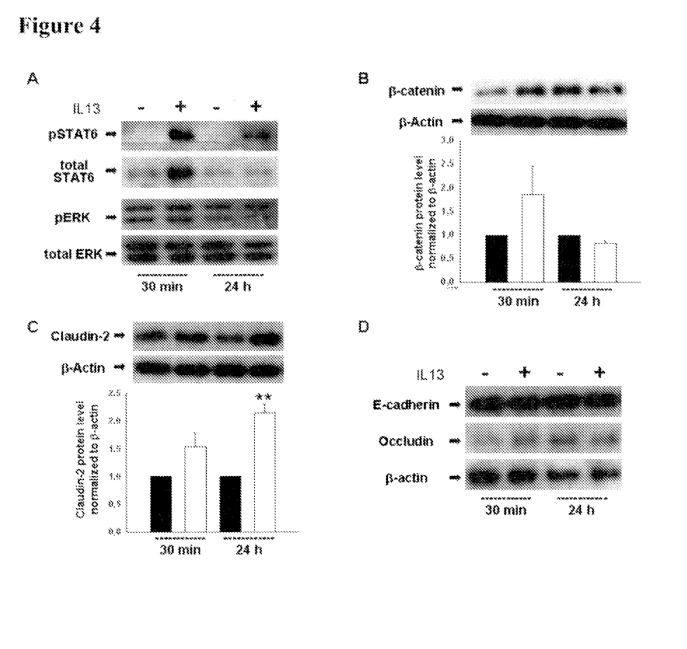

We next assessed, whether IL-13 could be involved in EMT-associated effects in IEC. HT29-IEC were treated with 100 ng/ml IL-13 for 30 min or 24 h, respectively. Administration of IL-13 induced mRNA levels of SLUG and β6-integrin by 24 h treatment (FIGS. 3A+B), but had no effect on SNAIL1, TGFβ and PTPN2 mRNA expression at any tested time point (FIGS. 3C-E). To study how IL-13 could affect β6-integrin levels, we performed SLUG knock-down studies using SLUG-specific siRNA constructs that caused a clear reduction in SLUG mRNA levels (data not shown). As before. IL-13 induced β6-integrin mRNA levels by 24 h treatment. This effect was, at least partially, diminished in SLUG knock-down cells (FIG. 3F). On a protein level, IL-13 induced the phosphorylation, indicative for activation, of the signalling intermediates, STAT6 and ERK1/2 by treatment for 30 min. IL-13 caused also an increase in levels of β-catenin, indicating increased signal transduction via this pathway, by treatment for 30 min (though we admit that this was statistically not significant) and after 24 h treatment levels of β-catenin protein were similar than those in unstimulated control cells (FIG. 48). While levels of the intercellular pore protein, claudin-2, were increased by 24 h IL-13 treatment (FIG. 4C), protein levels of the intercellular adhesion molecule, E-cadherin, as well as of the tight junction molecule, occludin, were not affected at that time point when compared to untreated control cells (FIG. 4D). These data indicate that IL-13 induces the expression of genes that are involved in cell invasion in HT29 IEC and might hereby contribute to the invasiveness of CD-associated fistulae that penetrate into the tissue.

### Example 4

### TGFβ, but not IL-13, Induces EMT in a HT29-Spheroid Cell Model

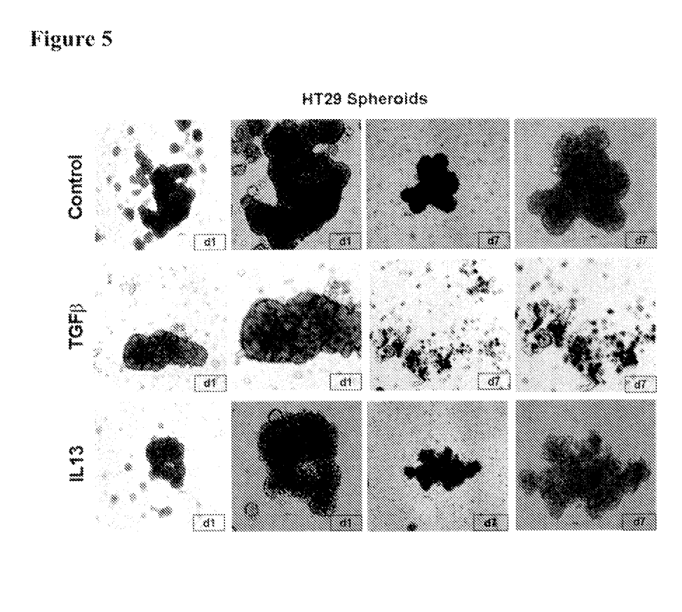

Since we had shown that IL-13 induces the expression of molecules associated with cell invasion, we next studied whether IL-13 would be able to induce a disintegration of a epithelial cell formation in an in vitro model of EMT. We seeded HT29 cells as spheroids for 7 d. Then, spheroids were either left untreated or treated with TGFβ (20 ng/ml) or IL-13 (100 ng/ml) for additional 7 d. By microscopy we observed the morphological development of the HT29-spheroids over time. All of the studied spheroids presented as compact cell formations after 7 d of seeding. Untreated HT29 spheroids showed a similar appearance even after 14 d with a clear black line indicating the boarder of the cell formation. In contrast, TGFβ treatment resulted in the almost complete disassembly of the cell formation after 7 d of growth factor treatment, indicative for the onset of EMT. IL-13-treated spheroids featured no obvious morphologic signs for the onset of EMT, since no disintegrated, single cells were detectable. However, the rim of the cell formation was somewhat diffusely confined when compared to untreated control spheroids (FIG. 5). These findings suggest that IL-13 alone is, in contrast to TGFβ, not sufficient to induce EMT in IEC.

### Example 5

### TGFβ Induces IL-13 and SNAIL1 mRNA Expression in HT29-Spheroids

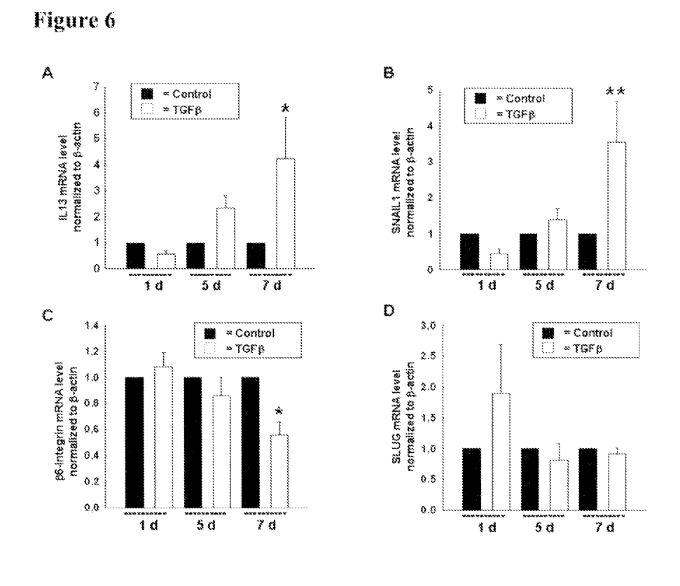

We next assessed alterations in gene expression pattern induced by TGFβ that could contribute to the induction of EMT in our cell model. We found that TGFβ treatment resulted in a time-dependent increase in IL-13 mRNA that reached statistical significance after 7 d of treatment (FIG. 6A). However, the concentration of IL-13 in the supernatant of these spheroids was below the detection level of our used ELISA system (data not shown). We also found that TGFβ increased mRNA expression of the EMT-associated transcription factor, SNAIL1, in these spheroids following a time-dependent kinetic and reaching a peak after 7 d (FIG. 6B). As shown above, at that time point, HT29 spheroids had completely disintegrated, indicative for the onset of EMT in these cells (FIG. 5). These findings also correlated with our observations in fistula specimen from CD patients showing that TC lining the fistula tracts, that are thus currently undergoing EMT, feature strong staining for IL-13 (FIG. 2A) as well as for SNAIL1 (Scharl, in press). In contrast, TGFβ treatment resulted in a significant decrease of β6-integrin expression after 7 d treatment (FIG. 6C) and did not (significantly) affect SLUG mRNA at any investigated time point (FIG. 6D). These data indicate that TGFβ induces EMT-related transcription events in HT29-spheroids and provide a further hint that the growth factor is responsible for the observed IL-13 expression in TC cells alongside CD fistula tracts.

### Example 6

### IL-13 Induces SLUG and β6-Integrin Expression in an In Vitro Model of EMT

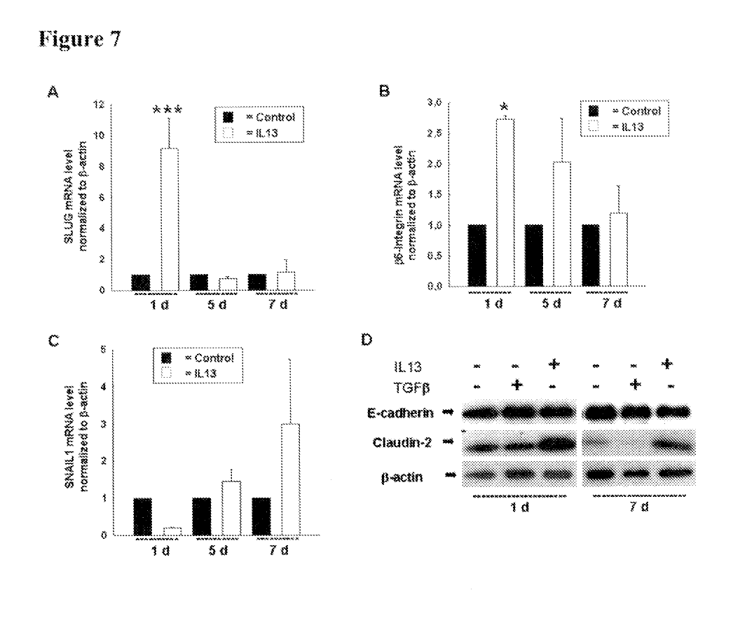

SLUG and β6-integrin were clearly detectable in TC and fistula surrounding cells of CD fistulae (6) (Scharl in press). Here, we have shown that IL-13 induces the expression of both of those genes in HT29 monolayers. Interestingly, in HT29-spheroids undergoing EMT, TGFβ stimulation was not sufficient to induce mRNA expression of SLUG and β6-integrin. We next studied whether IL-13 could induce their mRNA levels in an EMT cell model. Treatment of HT29 spheroids with IL-13 induced SLUG mRNA levels up to 9-fold already after incubation for 1 d. After 5 d and 7d, SLUG expression levels were comparable to those in untreated control cells (FIG. 7A). A similar finding was observed for β6-integrin. IL-13 induced its gene expression by 1 d incubation and mRNA levels declined over time to a level equally to those in control cells (FIG. 78). Of notice, IL-13 treatment decreased mRNA levels of SNAIL1 after 1 d, but resulted in increased SNAIL1 mRNA expression after 7 d treatment, indicating an opponent IL-13-induced expression pattern for SNAIL1 than that observed for SLUG and β6-integrin (FIG. 7C). On a protein level, TGFβ and IL-13 treatment resulted in a slight reduction of E-cadherin levels after 7 d treatment. While TGFβ administration did not affect claudin-2 protein. IL-13 caused, as expected, a strong increase in claudin-2 protein levels after already after 1 d and also after 7 d treatment (FIG. 7D). These data further support the hypothesis that TGFβ induces EMT-specific gene expression pattern in IEC, whereas IL-13 induces the expression of genes associated with cell invasion.

### Example 7

### Levels of SLUG, β6-Integrin and MMP-13 are Increased in CD Fistulae CLPF

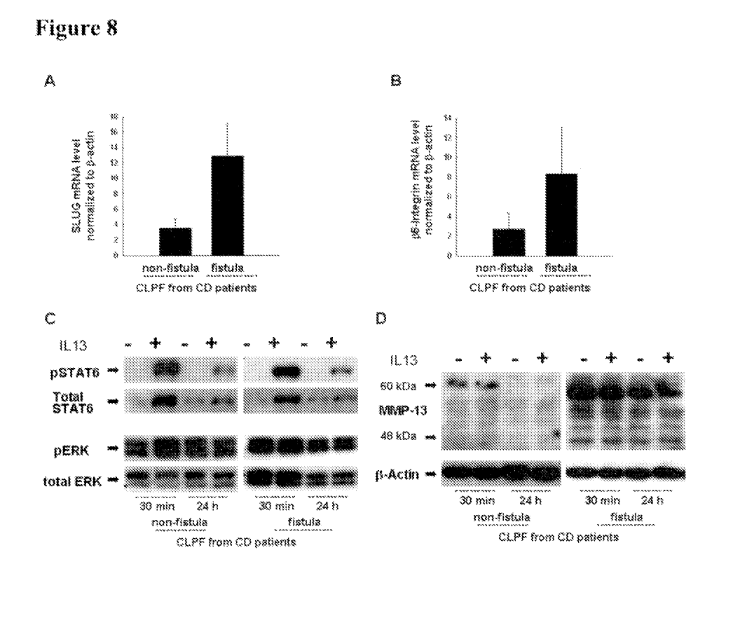

Since we had shown that IL-13 affects SLUG and β6-integrin gene expression in IEC, we tested whether the basal expression of these genes would be different in CLPF isolated from patients with fistulising CD when compared to CLPF isolated from patients with non-fistulising CD. We found that SLUG mRNA levels were about 4-fold higher in CLPF derived from patients with fistulising CD than in CLPF from patients with non-fistulising CD (FIG. 8A). A similar finding was obtained for β6-integrin mRNA levels. (FIG. 8B). We then treated our CLPF with IL-13 to study potential differences in the responsiveness of these cells to the cytokine. However, at the studied time points no differences in the ability of IL-13 to stimulate the phosphorylation of the signalling intermediates. STAT6 and ERK1/2, could be detected. Additionally, no obvious differences in their baseline phosphorylation could be observed (FIG. 8C). We then investigated baseline as well as IL-13-induced levels of full-length and cleaved MMP-13 (collagenase 3). The cleaved isoform of MMP-13 represents the activated protein, which is involved in ECM degradation and associated with tumor metastasis, in example of breast cancer (29). Basal levels of full length as well as cleaved MMP-13 were clearly higher in fistula CLPF than in CLPF from patients with non-fistulising disease. However, IL-13 treatment did not cause any obvious alterations in levels of both of the MMP-13 isoforms (FIG. 8D). These data demonstrate that CLPF derived from CD patients with fistulising disease exhibit higher levels of molecules associated with cell invasion than CLPF from patients with non-fistulising disease.

Discussion
We have demonstrated that IL-13 is detectable in TC lining fistula tracts and in IEC of deformed crypts adjacent to CD-associated fistulae. TGFβ, the most powerful inducer of EMT, was capable to induce IL-13 secretion from CLPF derived from CD patients with fistulising disease. In an EMT cell model using HT29 IEC, TGFβ also induced IL-13 mRNA by chronic exposure. On a functional level, IL-13 caused increased expression of genes associated with cell invasion, indicating a role for IL-13 in the pathogenesis of CD fistulae.
As we have shown previously TC lining the tracts of CD-associated fistulae feature several aspects that strongly support the onset of EMT. In particular they express high levels of transcriptionally active SNAIL1, a downregulation of E-cadherin as well as the concomitant expression of epithelial (cytokeratine-8 and 20) as well as mesenchymal markers (vimentin). Additionally, considerable levels of TGFβ are detectable in TC lining the fistula tracts (6). A further hint to the onset of EMT during fistula pathogenesis is the fact that fistula CLPF strongly upregulate SNAIL1 mRNA levels, which could not be observed in CLPF derived from CD patients without fistulae.
Here, we demonstrated strong staining for IL-13 and its receptor, IL-13Rα₁, in TC cells lining the fistula tracts as well as in epithelial cells of deformed crypts adjacent to the fistulae. This observation was somewhat unexpected, since IL-13 was thought to be mainly expressed by immune cells, especially Th₂ cells (18). Though IL-13 has not been associated with EMT so far, it has been clearly correlated with the onset of tissue fibrosis, such pulmonary fibrosis, hepatic fibrosis or systemic sclerosis (20-21, 30). We showed high levels of IL-13 in cells that feature invasive and penetrating cell growth into vicinal tissue layers, such as TC. Interestingly, we also found high levels of IL-13Rα₁, suggesting that IL-13 itself causes effects on these cells in an autocrine manner.
We then investigated the possible driving force for the expression of IL-13 in TC or IEC, respectively. By stimulating CLPF cultures with TGFβ, we found that the growth factor induced the secretion of IL-13 from fistula CLPF, but not from non-fistula CLPF. Additionally, CLPF isolated from patients with fistulising CD featured also higher basal levels of IL-13 secretion than cells from CD patients without fistulae. By Western blotting, we found that TGFβ-treated fistula CLPF exhibit reduced levels of PTPN2, which is contrary to the elevated IL-13 secretion from these cells. TNF, that has been widely shown to play a pivotal role for CD pathogenesis (31), was not sufficient to further elevate IL-13 secretion from fistula CLPF, but strongly induced PTPN2 expression. The suggested regulatory role for PTPN2 with respect to IL-13 secretion could be further defined using IEC. Here TNF was, similar than in fistula CLPF, not sufficient to induced IL-13 secretion in PTPN2-competent cells. However, PTPN2 knock-down allowed TNF to induce IL-13. Though TGFβ decreased PTPN2 protein levels in IEC, it was still not sufficient to induce IL-13 secretion from these cells after 48 h treatment, but did so after 7 d treatment. This indicates that a certain basal of functional PTPN2 is able to prevent IL-13 secretion in IEC. These observations are of special interest, since a single nucleotide polymorphism in the PTPN2 gene has recently been associated with a penetrating CD disease course (32). Nevertheless, additional events seem to be necessary to allow TGFβ to stimulate the secretion of IL-13 from IEC, possibly the concomitant expression of SNAIL1 transcription factor or even epigenetic modifications in these cells following long-term exposure to TGFβ (though we admit that this has not been formally demonstrated). However, all in all, our data suggest that PTPN2 activity is capable of controlling IL-13 secretion in IEC and CLPF and reveal a functional aspect, how genetic PTPN2 variants could contribute to the onset of a penetrating CD phenotype.
The TC represent originally IEC that underwent EMT. However, in addition to their transformatory potential, they obviously exhibit a considerable ability to penetrate into adjacent tissue layers, since they can be found at the invasive top of the fistulae. We have previously found that SLUG and β6-integrin are expressed in TC or mesenchymal-like cells around the fistula tracts (Scharl, in press) (6). Both of these genes have been associated with tumour invasiveness and cell invasion (15-17). Therefore, we speculated that IEC should upregulate the expression of both of these molecules during EMT course. However, though chronic treatment of HT29 spheroids with TGFβ resulted in an EMT-like disintegration of the epithelial cell formation and the EMT-typical upregulation of SNAIL1 mRNA, it was not sufficient to induce mRNA expression of SLUG or β6-integrin, but resulted in increased mRNA levels of IL-13. Since we had found strong staining for IL-13 and IL-13Rβ₁ in TC as well as elevated levels of SLUG and β6-integrin in CLPF from patients with fistulising CD when compared to those from non-fistulising CD, we hypothesized that IL-13 could act on TC in an autocrine manner to induce the expression of genes associated with cell invasion. To test this assumption, we treated HT29 IEC with IL-13. We found that the cytokine was able to induce mRNA levels of SLUG and β6-integrin in HT29 monolayers as well as in the spheroid model. However, though in the latter one, IL-13 was not sufficient to induce an EMT phenotype of these cells, these observations are in good line with recent findings showing that IL-13 is associated with increased cell invasion in pancreatic cancer (23-26). Altogether, these observations strongly support a previously unknown role for IL-13 in the pathogenesis of CD-associated fistulae.
TGFβ treatment of HT29 spheroids resulted in a time-dependent upregulation of SNAIL1 mRNA, but downregulation of β6-integrin and SLUG, reaching a peak for all described effects after 7 d. Vice versa, IL-13 induced SLUG and β-integrin levels already after 1 d of treatment and their expression levels declined to control levels after 7 d.

Interestingly, SNAIL1 mRNA expression was three-fold higher after 7 d IL-13 treatment than in control cells. These observations suggest that SNAIL1 could act as a repressor of SLUG and β6-integrin expression in IEC. This findings could make sense in a way that TGF□/SNAIL1-induced EMT acts as a mechanism of wound healing and tissue regeneration at sites of chronic inflammation and tissue destruction, as present during active CD (33), but does not feature an invasive potential which can be observed in CD fistulae. In contrast, IL-13, which is expressed after chronic exposure of IEC to TGFβ, drives the invasive potential of EMT cells in an autocrine manner. This observation seems to be in contrast of previous findings, since during fibrogenesis, IL-13 acts upstream of TGFβ, whereas in the setting of cell invasion, the cytokine seems to be regulated by the growth factor.
In summary, our data show for the first time a functional role for TGFβ and IL-13 in the pathogenesis of CD-associated fistulae. Both of the mediators seem to collaborate in a synergistic step-by-step process whereby TGFβ induces EMT by causing disintegration of the epithelial cell formation and IL-13 finally enables the EMT cells to penetrate into deeper tissue layers. These findings suggest that a dysregulation of TGFβ and/or IL-13-induced effects plays a pivotal role for the pathogenesis of CD-associated fistulae and emphasize the important of further investigations of the detailed mechanisms resulting in the onset of fistulae during CD course. In addition these observations might open new avenues for the development of new and more effective therapeutic strategies for the treatment of such fistulae.

### Material & Methods for Examples 1-7

Human IL-13 (R&D Systems, Abingdon, UK), TGFβ (Calbiochem, San Diego Calif.), TNF (Calbiochem), mouse anti-β-actin (Sigma, St. Louis, Mo.), mouse anti-PTPN2 (Calbiochem), mouse anti-claudin-2 (Invitrogen, Carlsbad, Calif.) and rabbit anti-ERK1/2 antibodies (Santa Cruz, Santa Cruz, Calif.) were obtained from the sources noted. Rabbit anti-phospho-ERK1/2-(Thr²⁰²/Tyr²⁰⁴), rabbit anti-STAT6, rabbit anti-phospho-STAT6-(Tyr⁵⁴¹), rabbit anti-β-catenin, rabbit anti-E-cadherin, rabbit anti-occludin antibodies were obtained from Cell Signaling Technologies, Danvers, Mass. Rabbit anti-MMP-13 (Abcam, Cambridge, Mass.) antibody detected both, full length and cleaved, protein variants. All other reagents were of analytical grade and acquired commercially.

Cell Culture
Human T₈₄ IEC were cultured in a humidified atmosphere with 10% CO₂ in 4.5% high glucose Dulbecco's modified Eagle's medium (Invitrogen, Carlsbad. CA) supplemented with 10% newborn calf serum. Human HT29 IEC were cultured in a humidified atmosphere with 10% CO₂ in McCoy's 5A Medium (JRH, Lenexa, Kans.) supplemented with 10% fetal calf serum. Cells were separated by trypsinization. When grown as a monolayer, 1×10⁶ cells were seeded onto 12 mm Millicell-HA semi-permeable filter supports (Millipore, Bedford, Mass.). Before treatment, cells were cultured for 5-7 days. According to their receptor localization, IL-13 (100 ng/ml), TNF (100 ng/ml) and TGF□ (50 ng/ml) were added basolaterally. For spheroids, 4500-5000 HT29 cells per well were seeded in a Terasaki plate (Greiner Bio-One, Frickenhausen, Germany) and grown for 7 d. Then cells were stimulated by adding IL-13 or TGFβ into the medium for further 7 d. Morphological development of spheroids was monitored on day 8, 10, 12 and 14 by transmission microscopy using an AxioCam MRc5 (Zeiss, Jena, Germany) on a Zeiss Axiophot microscope (Zeiss) with AxioVision Release 4.7.2 software (Zeiss).

Patient Samples
Perianal fistulae specimens from CD patients for immunohistochemistry were prospectively collected from male and female individuals with and without CD. We investigated seven fistulae in formalin-fixed tissue samples from seven CD patients. Fistulae were surgically resected and immediately transferred into 4% formalin and stored at 4° C. until further analysis. Primary CLPF cultures were obtained from fistulising areas of the intestinal mucosa of 7 CD patients (mean age 53±5 years) or from the intestinal mucosa of 5 patients with non-fistulising CD (mean age 45±13 years). Samples were collected from male and female patients and CLPF cultures were collected from surgical specimens. Written informed consent was obtained before specimen collection and studies were approved by the local ethics committee.

Isolation and Culture of Human CLPF
Procedures were performed as described previously (34). While biopsies were used immediately for isolation of CLPF, the mucosa from surgical specimens was initially cut into 1 mm pieces and washed in fibroblast medium consistent of Dulbecco's Modified Eagle's high glucose medium (DMEM; PAA, Cölbe, Germany) containing 10% fetal calf serum (FCS; Gibco, Karlsruhe, Germany) and cultured in 25 cm² culture flasks (Costar, Bodenheim, Germany) with DMEM containing 10% FCS, penicillin (100 IU/ml), streptomycin (100 μg/ml), ciprofloxacin (8 μg/ml), gentamycin (50 μg/ml) and amphotericin B (1 μg/ml). The tissue was rinsed and digested for 30 min at 37° C. in phosphate buffered saline (PBS, Gibco,) containing Ca²⁺ and Mg²⁺ (PAA), 1 mg/ml collagenase I (Sigma, St. Louis, Mo.), 0.3 mg/ml DNase I (Boehringer, Mannheim, Germany) and 2 mg/ml hyaluronidase (Sigma). After isolation cells were rinsed with Removal of non-adherent cells was performed by multiple medium changes and remaining cells were used between passages 3 and 8.

Stimulation of Human CLPF
2×10⁸ cells were seeded as described previously and cultured for 24 h at 37° C. (34). Medium was removed, cells were washed twice with PBS and IL-13 (100 ng/ml) or TGFβ (50 ng/ml) were applied in DMEM without FCS. Control cells were kept in serum free media (DMEM without FCS), respectively.

Immunohistochemistry
Immunohistochemical studies were performed on formalin-fixed, paraffin-embedded tissue specimen using a peroxidase based method with diaminobenzidine (DAB) chromogen as described previously²³. In brief, tissue samples were incubated with xylol and descending concentrations of ethanol. Antigen retrieval was performed using citrate buffer, pH 6.0 (DAKO, Glostrup, Denmark) for 30 min at 98° C. Endogenous peroxidases were deleted by incubation with 0.9% hydrogen peroxide for 15 min at room temperature (RT) and blocking was performed using 3% BSA for 1 h at RT. Antibodies were then applied in an optimal concentration over night in a wet chamber. Rabbit anti-IL-13 (R&D Systems) and IL-13Rα₁ (Abcam) antibodies were obtained from the sources noted. Secondary antibody (EnVision⁺ System-HRP-Labelled Polymer from DAKO) was applied for 1 h at RT and antibody binding was visualized by a Liquid DAB+ Substrate Chromogen System (DAKO). Then samples were counterstained with hematoxylin, incubated in ascendingly concentrated ethanol and xylol solutions and finally mounted. Microscopic assessment was done using an AxioCam MRc5 (Zeiss, Jena, Germany) on a Zeiss Axiophot microscope (Zeiss) with AxioVision Release 4.7.2 software (Zeiss).

Real-Time PCR

Cells were resuspended in RLT-buffer (Qiagen, Valencia, Calif.) and lysed using a 24-gauge needle on a syringe. mRNA was isolated using RNeasy Plus Mini Kit (Qiagen, Valencia, Calif.) using a QIA-Cube (Qiagen) including shredder and DNA was removed by DNase I (Qiagen) according to manufacturer's instructions. RNA concentration was assayed by absorbance at 260 and 280 nm content using NanoDrop ND1000 (Thermo Scientific). cDNA synthesis was performed using a High-Capacity cDNA Reverse Transcription Kit (Applied Biosystems, Foster City, Calif.). Real-time PCR TaqMan Assays and TaqMan Gene Expression Master Mix were obtained from Applied Biosystems. Real-time PCR was performed on a 7900HT Fast Real-Time PCR System using SDS 2.2 Software (Applied Biosystems). Triplicate measurements were performed and human β-actin was used as endogenous control. Results were then analyzed by ΔΔCT-method. The real-time PCR contained 40 cycles.

Preparation of Whole Cell Lysates
Whole cell lysates were generated using M-Per Mammalian protein extraction reagent (Pierce Biotechnology, Rockford. IL) according to manufacturer's instructions. Protein content was measured using NanoDrop ND1000 (Thermo Scientific).

Western Blotting
An aliquot of each lysate was mixed with NuPAGE® 4×LDS Sample Buffer (Invitrogen) and 50 mM dithiothreitol (Sigma) and boiled for 5 min at 96° C. Proteins were separated by SDS-polyacrylamide gel electrophoresis and transferred onto nitrocellulose membranes (Invitrogen). Membranes were blocked with 1% blocking solution and rabbit anti-SNAIL1 antibody (1:1000; Abcam) was applied over night. Membranes were washed with Tris buffered saline containing 1% Tween 20 (1% TBST) for 1 h, HRP-labelled secondary anti-rabbit-IgG-antibody (1:3000; Santa Cruz) was added for 30 min and membranes were washed for 1 h with 1% TBST. Finally, immunoreactive proteins were detected using an enhanced chemiluminescence detection kit (GE Healthcare, Little Chalfont, UK).

ELISA
Cell supernatant was collected and stored at −80° C. until further treatment. ELISA Kits detecting human IL-13 were obtained from Promokine (Heidelberg, Germany). Assays were performed according to manufacturer's instructions using a sample volume of 50 μl per reaction. Absorbance at 450 nm was detected on a BioTek Synergy 2 Microplate reader using Gen 5 Software (BioTek Instruments, Inc., Winooski, Vt.). Measurements were performed in duplicate.

SiRNA Transfection
HT29 (2×10⁶) cells were seeded 5 days before transfection. 100 pmol of three different annealed SLUG-specific Silencer Pre-designed siRNA oligonucleotides (Applied Biosystems) were transfected into HT29 cells using the Amaxa nucleofector system (Lonza, Walkersville, Md.). After transfection, IEC were cultured on filter membranes for 48 h before treatment. Non-specific control siRNA (ABI) (100 pmol/transfection) was used as negative control.

Statistical Analysis
Data are presented as means+/−S.E.M. for a series of n experiments. Statistical analysis was performed by ANOVA followed by Student-Newman-Keuls post hoc test ot Student's t-test, where appropriate. P values <0.05 were considered significant.

### Example 8

### Treatment of Fistula in CD Patients

This example shows how the treatment of fistula in CD patients with a particular antibody can be carried out and the effects assessed. It will be obvious to those skilled in the art that other IL-13 antibodies may be used in a similar manner although for example the exact dosing and administration schedule may vary dependent on the particular antibody used.

### Rationale of Dose/Regimen, Duration of Treatment

Efficacy in the proposed trial will require changes in fistulas leading to their closure. The current thinking hypothesizes that high local expression of IL-13 in the cells lining the fistula tracts is a key mediator of tissue remodeling and fibrosis. Thus, to achieve an effect on closure inhibition of local IL-13 in the fistula tissue is necessary. The required amount of 01951/G12 in the fistulas to inhibit IL-13 can be in principle derived from the equation describing the binding equilibrium between 01951/G12 and IL-13. However, the local IL-13 concentrations are not known. In addition, the fistula tissue accessibility for 01951/G12 is unknown, thus local 01951/G12 concentrations can not be predicted from plasma concentrations. As a consequence, sufficiently high doses should be used to drive permeation of 01951/G12 into fistula tissue. To give 01951/G12 the best chance to inhibit local IL-13, it is proposed to dose 10 mg/kg of 01951/G12 every 3 weeks.

### 01951/G12 150 mg Powder for Solution, in Glass Vials.

01951/G12 150 mg Powder for Solution will be provided in glass vials each containing 150 mg 01951/G12 as a lyophilized cake. The vials contain a 20% overfill to allow a complete withdrawal of the labeled amount of 01951/G12. The manufacturing process for 01951/G12 Powder for Solution consists of standard manufacturing processes: Dilution, mixing/stirring, pre and sterile filtrations, aseptic filling and lyophilization. The drug product is considered to be stable until the date indicated on the drug product label if stored at 2 to 8° C. Based on results of ongoing stability studies the re-test period will be adjusted as appropriate. Immediately prior to administration, Sterile Water for Injection (SWFI) is added to the vial and the powder is dissolved ready for use to yield a single-use Concentrate for Solution for Infusion which needs to be diluted before use. For subsequent intravenous administration, the yielded Concentrate for Solution for Infusion needs to be further diluted to the ready-to-use 01951/G12 Solution for Infusion.

### 01951/G12 Solution for Infusion

Reconstitution of 01951/G12 150 mg Powder for Solution with 1.0 mL SWFI will produce a Concentrate for Solution for Infusion at a concentration of 150 mg/ml 01951/G12 in a total final volume of 1.2 ml. The Concentrate for Solution for Infusion is available as histidine (pH 6.0±0.5) buffered solution, containing sucrose, glycine and polysorbate 80. The formulation does not contain a preservative as it is to be used for single-dose administration only. This concentrate is subsequently diluted in an infusion bag containing 5% glucose/dextrose solution in accordance with the instructions for use provided below. Since 01951/G12 is a protein, the reconstituted vials may contain a few translucent particles. The Solution for Infusion must therefore be infused through a 0.2 micron in-line filter (see filter supplier requirements under “Materials to be used”).
Do NOT directly inject the Concentrate for Solution for Infusion into subjects, but follow the preparation instructions below.

### Dose/Volume Calculation

Note: The vials contain an overfill of 20% of 01951/G12. The dose/volume calculations as described below must be strictly adhered to.

The dose for administration to subjects will be calculated from the individual subjects' body weight as measured at the baseline visit.

Dose levels of 10 mg/kg can be administered.
Dose calculation:
Dose (mg)=weight of patient (kg)×dose level (mg/kg).
Volume calculation:

To obtain the volume of 01951/G12 Concentrate for Solution for Infusion which is needed, the calculated dose is to be divided by the concentration of the Concentrate for Solution for Infusion (i.e. 150 mg/mL)

### Example

Subject weight: 65 kg

Dose level: 10 mg/kg

Calculated dose: 650 mg

Calculated volume: 650 mg/150 mg/mL=4.3 mL

Calculated number of vials: 4.3/1.2=4 per dose

### Materials to be Used:

The infusion set including the intravenous filter set has to be prepared according to the instructions supplied by the manufacturers (no product reference numbers are given as they might be country-specific):
Infusion bags:
Baxter Viaflex 5% Dextrose Injection 250 mL
B. Braun Ecoflac plus 5% Glucose, 250 mL
Infusion lines:
Baxter Solution Set 101′ (2.6 m) with Male Luer Lock Adapter
B. Braun Original Infusomat tubing, type standard (250 cm)
Alaris Pump module administration set: Low sorbing set with 0.2 micron (low protein binding) filter and 1 injection port (285 cm)
In-Line filter (must be attached if using the Baxter or B. Braun infusion lines):
Pall Posidyne® ELD 0.2 μm Intravenous Filter Set
Sufficient 01951/G12 150 mg powder for solution vials to dispense the calculated dose (see above under Dose).
1 mL graduated disposable syringes to perform the reconstitution of the lyophilisate (to measure 1.0 mL).
Syringes of appropriate volume for performing the dilution.
Needles of suitable size (e.g. 21 G×2″) for reconstitution and withdrawal of the reconstituted solution.
Sterile Water for Injection (SWFI).

### Preparation

Reconstitute each vial by slowly injecting 1.0 mL of SWFI into the vial containing the lyophilized cake of 01951/G12. The stream of diluents should be directed onto the lyophilized cake. Then the vial is tilted by an angle of approx. 45° and gently rotated between the fingertips for approx. 1 minute.
Thereupon the vial is incubated standing on the bench at room temperature for a minimum
   of 10 minutes with occasional rotating the vial as described above (3-5 times/15 minutes, 20-60 seconds each). Note that foaming of the solution is not unusual. Do not shake or invert the vial.
Allow the vial to stand undisturbed for approximately 5 minutes. The resulting solution should be essentially free of visible particles, clear to opalescent, and colorless.
Calculate the volume of 01951/G12 Concentrate for Solution for Infusion to be used for each individual subject according to body weight and dose level (see above under Dose/Volume calculation).
Withdraw and discard this volume from the infusion bag.
Carefully withdraw the calculated volume of 01951/G12 Concentrate for Solution for Infusion from the vials into a suitable syringe.
Inject the 01951/G12 Concentrate for Solution for Infusion slowly into the infusion bag and mix by agitating the bag gently. Do not shake to avoid foaming.

### Administration

01951/G12 should be administered as an infusion at a flow rate of about 2 mL/min (total administration time: approximately 120 minutes) using materials specified above (see Preparation of the infusion bags). 01951/G12 infusion can be performed by gravitational way of administration or using infusion pumps (i.e. Colleague CXE volumetric infusion pump if using the Baxter infusion line; Infusomat® fmS volumetric infusion pump if using the B.

Braun Infusomat infusion line, Alaris Gemini infusion pump if using the Alaris Pump module infusion line).

### Clinical Assessment of Fistula Activity

Fistula closure will be clinically assessed by the investigator. Clinical assessment of fistula activity includes assessment and documentation of•Location and appearance of fistula(s) with description of indurations, color and estimation of area of cutaneous fistula opening(s);
Amount of purulent fistula discharge after exertion of gentle pressure on fistula edges. In addition, it may be assessed if fistula activity is increased, unchanged or reduced in comparison to the previous visit.

### Photodocumentation

Photodocumentation will be utilized during this study to allow for documentation of fistula healing.

### MRI Assessment

MRI is a useful technique to study the pelvis because it offers excellent soft tissue discrimination with a wide field of view, and it is free of radiation hazard. In this study, pelvis MRI will be used to assess the complexity and behavior of perianal fistulas over time. MRI images will be analyzed to produce a score reflecting both anatomical changes and active inflammation around the fistula tracks, as described by (Van Assche, et al 2003).

### Health-Related Quality of Life (SIBDQ)

The purpose of including the Short Inflammatory Bowel Disease Questionnaire (SIBDQ) in this study is to assess disease-specific health related quality of life in subjects with fistulizing Crohn's disease. The SIBDQ is a valid, sensitive and reliable measure used extensively in clinical trials and research. The SIBDQ comprises 10 items, which are grouped into four subscales, including: bowel symptoms, systemic symptoms, emotional function, and social function. Each item is scored on a 7-point Likert scale ranging from 1 (worst) to 7 (best).

### Endoscopic Biopsy Procedure

Biopsies from the fistula tracts will be obtained endoscopically during screening and 1 week after the first application of 01951/G12 (D8±2 days). Aim is to obtain biopsies from the lining of the fistula tracts via their luminal opening. In case the luminal fistula opening is inaccessible, the investigator will seek sponsor's advice and mutual agreement on a per case basis how to proceed. In such cases it is e.g. conceivable that mucosal biopsies are being obtained from the immediate vicinity of the internal fistula opening.

The choice of endoscopic technique is at the discretion of the investigators; most patients will have to be appropriately analgo-sedated for the biopsy procedures. Under visual control, the investigator will take 1 to 2 standard biopsies from the wall of the fistula tract. One biopsy will be immediately placed into RNA preservation medium and the other biopsy will be placed in a tube containing 10% buffered formalin solution and processed for histological examination. The procedure will be further detailed in a separate guidance sheet.

The samples processed for histology will be examined by a pathologist to evaluate the morphological changes in the wall of the fistula tract and the remaining paraffin block will be saved for further investigations (immunohistochemistry and/or in situ hybridization).

The sample intended for gene expression profiling will be processed for RNA microarray analysis.

### Soluble Biomarkers Including but not Limited to:

TGF-β, periostin, eotaxin-1, PICP and PIIINP, IL-4

### Rational

Serum and plasma samples will be collected to evaluate downstream biomarkers of the IL-13 pathway or in relation to other fibrotic mechanisms. The final biomarker panel will include, but will not be limited to TGF-β, periostin, eotaxin-1, PICP, PIIINP and IL-4. To be able to evaluate further biomarkers like IL-13 receptors depends on the availability of related assays.

The potential relationship between biomarker and clinical responses will be explored.

The question of whether any specific biomarkers (or combination of them) at certain levels are more important predictors of clinical response to treatment than others will be investigated to allow better definition of the target population. Additional blood for plasma (4 mL per time point) is taken for potential further investigation.

### Sample Collection Procedure for TGF-β, Periostin, Eotaxin-1, PICP and PIIINP

Serum

A single 14 ml blood sample will be drawn to ensure 9 ml serum.

All blood samples will be taken by either direct venipuncture or an indwelling cannula inserted in a forearm vein and collected into a sterile tube. After blood collection, the blood sample is allowed to clot for 30 min at room temperature. The tube must then be placed on ice. Samples should be then centrifuged immediately at 2000×g for 10 min at 4° C. After centrifugation, the supernatant is transferred to a new sterile polypropylene tube and gently mixed by inversion.

Serum-samples are aliquoted at 250 μl in 0.5 ml polypropylene cryovials (Sarstedt No. 72.730.006 or equivalent) and frozen immediately at least at −20° C. (=−70° C. is the preferred temperature, however, samples should be treated equally) within 45 minutes of venipuncture. Shipment must be performed on dry ice on the same day as collection. Upon arrival in the central lab and the site of analysis, samples should be stored at=−70° C.

Except for clotting, leaving samples at room temperature during processing (even if only for a few minutes) should be avoided. Leaving samples on ice (2-4° C.) during processing should not exceed a total of 45 minutes. Samples should be shipped as specified in the laboratory manual.
Plasma

A single 4 ml venous blood sample should be collected in an EDTA tube to ensure 2 mL of plasma.

Immediately after each tube of blood is drawn, it should be inverted gently several times to ensure the mixing of tube contents (e.g., anticoagulant). Avoid prolonged sample contact with the rubber stopper. Place the tube upright in a test tube rack surrounded by ice until centrifugation. Within 30 minutes, centrifuge the sample at between 3 and 5° C. for 10 minutes at approximately 2500 g (or sufficient settings to achieve a clear plasma layer). Immediately after centrifugation transfer the supernatant plasma to 250 μl aliquots in 0.5 ml polypropylene cryovials (Sarstedt No. 72.730.006 or equivalent) and frozen immediately at least at −20° C. (−70° C. is the preferred temperature, however, samples should be treated equally) within 45 minutes of venipuncture. Shipment must be performed on dry ice on the same day as collection. Upon arrival in the central lab and the site of analysis, samples should be stored at −70° C.

### Fecal Calprotectin and Lactoferrin Levels

Fecal calprotectin and lactoferrin levels are broadly used biomarkers for the assessment and follow-up of the Crohn's Disease activity and correlate with endoscopic findings and will provide a non invasive, inflammatory disease marker.

### Sample Collection Procedure

For each scheduled sampling time point, two fecal samples (each approx. 5 g) will be collected into two 30 mL stool collection tubes, which are immediately stored at −18° C. to −20° C. The samples can be shipped on dry ice to the Central Lab with the next available shipment.

### Sample Analytical Methods

Changes in levels of fecal calprotectin and lactoferrin as non-invasive, inflammatory disease markers by ELISA will be explored with regard to their relationship to clinical efficacy.

### Preliminary Assessment of Clinical Study

A preliminary assessment of biological responses to the study treatment described above, showed that a patient with fistula responded to treatment with the 01951/G12 IL-13 antibody. The overall fistula activity in the patient decreased; specific improvements observed included pain reduction and cessation of mucupurulent discharge from fistula. The conclusion is that the IL-13 antibody had a positive clinical effect, reducing the fistula activity.

It will be understood that the invention has been described by way of example only and modifications may be made whilst remaining within the scope and spirit of the invention.

### REFERENCES

1. Solomon, M. J. 1996. Fistulae and abscesses in symptomatic perianal Crohn's disease. Int J Colorectal Dis 11:222-226.
2. Michelassi. F., Stella, M., Balestracci, T., Giuliante, F., Marogna, P., and Block, G. E. 1993. Incidence, diagnosis, and treatment of enteric and colorectal fistulae in patients with Crohn's disease. Ann Surg 218:660-666.
3. Schwartz, D. A., Loftus, E. V., Jr., Tremaine, W. J., Panaccione, R., Harmsen, W. S., Zinsmeister, A. R., and Sandborn, W. J. 2002. The natural history of fistulizing Crohn's disease in Olmsted County, Minnesota. Gastroenterology 122:875-880.
4. Bourikas, L. A., and Koutroubakis, I. E. 2010. Anti-TNF and fistulizing perianal Crohn's disease: use in clinical practice. Curr Drug Targets 11:187-197.
5. Judge, T. A., and Lichtenstein, G. R. 2004. Treatment of fistulizing Crohn's disease. Gastroenterol Clin North Am 33:421-454, xi-xii.
6. Bataille, F., Rohrmeier, C., Bates, R., Weber, A., Rieder, F., Brenmoehl, J., Strauch, U., Farkas, S., Furst, A., Hofstadter, F., et al. 2008. Evidence for a role of epithelial mesenchymal transition during pathogenesis of fistulae in Crohn's disease. Inflamm Bowel Dis 14:1514-1527.
7. Kalluri, R., and Neilson, E. G. 2003. Epithelial-mesenchymal transition and its implications for fibrosis. J Clin Invest 112:1776-1784.
8. Arias, A. M. 2001. Epithelial mesenchymal interactions in cancer and development. Cell 105:425-431.
9. Thiery, J. P., and Sleeman, J. P. 2006. Complex networks orchestrate epithelial-mesenchymal transitions. Nat Rev Mol Cell Biol 7:131-142.
10. Zavadil, J., and Bottinger, E. P 2005. TGF-beta and epithelial-to-mesenchymal transitions. Oncogene 24:5764-5774.
11. Kasai, H., Allen, J. T., Mason, R. M., Kamimura, T., and Zhang, Z. 2005. TGF-beta1 induces human alveolar epithelial to mesenchymal cell transition (EMT). Respir Res 6:56.
12. Margetts, P. J., Bonniaud, P., Liu, L., Hoff, C. M., Holmes, C. J., West-Mays, J. A., and Kelly, M. M. 2005. Transient overexpression of TGF-{beta}1 induces epithelial mesenchymal transition in the rodent peritoneum. J Am Soc Nephrol 16:425-436.
13. Bates, R. C., and Mercurio, A. M. 2003. Tumor necrosis factor-alpha stimulates the epithelial-to-mesenchymal transition of human colonic organoids. Mol Biol Cell 14:1790-1800.
14. Peinado, H., Quintanilla, M., and Cano, A. 2003. Transforming growth factor beta-1 induces snail transcription factor in epithelial cell lines: mechanisms for epithelial mesenchymal transitions. J Biol Chem 278:21113-21123.
15. Bates, R. C. 2005. Colorectal cancer progression: integrin alphavbeta6 and the epithelial-mesenchymal transition (EMT). Cell Cycle 4:1350-1352.
16. Bates, R. C., Bellovin, D. I., Brown, C., Maynard. E., Wu, B., Kawakatsu, H., Sheppard, D., Oettgen, P., and Mercurio, A. M. 2005. Transcriptional activation of integrin beta6 during the epithelial-mesenchymal transition defines a novel prognostic indicator of aggressive colon carcinoma. J Clin Invest 115:339-347.
17. Alves, C. C., Carneiro, F., Hoefler, H., and Becker, K. F. 2009. Role of the epithelial-mesenchymal transition regulator Slug in primary human cancers. Front Biosci 14:3035-3050.
18. Wynn, T. A. 2003. IL-13 effector functions. Annu Rev Immunol 21:425-456.
19. Donaldson, D. D., Whitters, M. J., Fitz, L. J., Neben, T. Y., Finnerty, H., Henderson, S. L., O'Hara, R. M., Jr., Beier, D. R., Turner, K. J., Wood, C. R., et al. 1998. The murine IL-13 receptor alpha 2: molecular cloning, characterization, and comparison with murine IL-13 receptor alpha 1. J Immunol 161:2317-2324.
20. Zhu, Z., Homer, R. J., Wang, Z., Chen, Q., Geba, G. P., Wang, J., Zhang, Y., and Elias, J. A. 1999. Pulmonary expression of interleukin-13 causes inflammation, mucus hypersecretion, subepithelial fibrosis, physiologic abnormalities, and eotaxin production. J Clin Invest 103:779-788.
21. Chiaramonte, M. G., Donaldson, D. D., Cheever, A. W., and Wynn, T. A. 1999. An IL-13 inhibitor blocks the development of hepatic fibrosis during a T-helper type 2-dominated inflammatory response. J Clin Invest 104:777-785.
22. Lee, C. G., Homer, R. J., Zhu, Z., Lanone, S., Wang, X., Koteliansky, V., Shipley, J. M., Gotwals, P., Noble, P., Chen, Q., et al. 2001. Interleukin-13 induces tissue fibrosis by selectively stimulating and activating transforming growth factor beta(1). J Exp Med 194:809-821.
23. Kornmann, M., Kleeff, J., Debinski, W., and Korc, M. 1999. Pancreatic cancer cells express interleukin-13 and −4 receptors, and their growth is inhibited by Pseudomonas exotoxin coupled to interleukin-13 and −4. Anticancer Res 19:125-131.
24. Fujisawa, T., Nakashima, H., Nakajima, A., Joshi, B. H., and Puri, R. K. 2010. Targeting IL-13Ralpha2 in human pancreatic ductal adenocarcinoma with combination therapy of IL-13-PE and gemcitabine. Int J Cancer.
25. Vallera, D. A., Stish, B. J., Shu, Y., Chen, H., Saluja, A. Buchsbaum, D. J., and Vickers, S. M. 2008. Genetically designing a more potent antipancreatic cancer agent by simultaneously co-targeting human IL-13 and EGF receptors in a mouse xenograft model. Gut 57:634-641.
26. Shimamura, T., Fujisawa, T., Husain, S. R., Joshi, B., and Puri, R. K. 2010. Interleukin 13 mediates signal transduction through interleukin 13 receptor alpha2 in pancreatic ductal adenocarcinoma: role of IL-13 Pseudomonas exotoxin in pancreatic cancer therapy. Clin Cancer Res 16:577-586.
27. Obiri, N. J., Husain, S. R., Debinski, W., and Puri, R. K. 1996. Interleukin 13 inhibits growth of human renal cell carcinoma cells independently of the p140 interleukin 4 receptor chain. Clin Cancer Res 2:1743-1749.
28. Serve, H., Oelmann, E., Herweg, A., Oberberg, D., Serve, S., Reufi, B., Mucke, C., Minty, A., Thiel, E., and Berdel, W. E. 1996. Inhibition of proliferation and clonal growth of human breast cancer cells by interleukin 13. Cancer Res 56:3583-3588.
29. Freije, J. M., Diez-Itza, I., Balbin, M., Sanchez, L. M., Blasco, R., Tolivia, J., and Lopez-Otin, C. 1994. Molecular cloning and expression of collagenase-3, a novel human matrix metalloproteinase produced by breast carcinomas. J Biol Chem 269:16766-16773.
30. Hasegawa, M., Fujimoto, M., Kikuchi, K., and Takehara, K. 1997. Elevated serum levels of interleukin 4 (IL-4), IL-10, and IL-13 in patients with systemic sclerosis. J Rheumatol 24:328-332.
31. Reinecker, H. C., Steffen, M., Witthoeft, T., Pflueger, I., Schreiber, S., MacDermott, R. P., and Raedler, A. 1993. Enhanced secretion of tumour necrosis factor-alpha, IL-6, and IL-1 beta by isolated lamina propria mononuclear cells from patients with ulcerative colitis and Crohn's disease. Clin Exp Immunol 94:174-181.
32. Morgan, A. R., Han, D. Y., Huebner, C., Lam, W. J., Fraser, A. G., and Ferguson, L. R. 2010. PTPN2 but not PTPN22 is associated with Crohn's disease in a New Zealand population. Tissue Antigens 76:119-125.
33. Rieder, F., Brenmoehl, J., Leeb, S., Scholmerich, J., and Rogler, G. 2007. Wound healing and fibrosis in intestinal disease. Gut 56:130-139.
34. Brenmoehl. J., Miller, S. N., Hofmann, C., Vogl, D., Falk, W., Scholmerich, J., and Rogler, G. 2009. Transforming growth factor-beta 1 induces intestinal myofibroblast differentiation and modulates their migration. World J Gastroenterol 15:1431-1442.
35. Dharmani and Chadee 2008. Biologic therapies against inflammatory bowel disease: a dysregulated immune system and the cross talk with gastrointestinal mucosa hold the key. Curr Mol Pharmacol: 1:195-212.
36. van Assche G, Vanbeckevoort D, Bielen D, et al. 2003; Magnetic resonance imaging of the effects of infliximab on perianal fistulizing Chrons disease Am J Gastroenterology 98:332-9

1: An anti-IL-13 antibody which inhibits or neutralizes the activity of IL-13 for use in the treatment or prevention of fistulas in patients suffering from Crohn's disease.

2. (canceled)

3: A method of treating or preventing formation of fistulas in patients suffering from Crohn's disease comprising administering an anti-IL-13 antibody which inhibits or neutralizes the activity of IL-13 to a subject in need thereof.

4: The method of claim 3 wherein the antibody comprises one or more of the CDRs selected from the list consisting of: (a) the V_{H} CDR1s shown in SEQ ID NOs: 1, 2, 6 or 7 (b) the V_{H} CDR2s shown in SEQ ID NOs: 3 or 8, (c) the V_{H} CDR3s shown in SEQ ID NOs: 4, 5, 9 or 10, (d) the V_{L} CDR1s shown in SEQ ID NOs: 11, 16, 17 or 18, (e) the V_{L} CDR2s shown in SEQ ID NOs: 12 or 19, (f) the V_{L} CDR3s shown in SEQ ID NOs: 13, 14, 15, 20, 21 or 22.

5: The method of claim 3 wherein the antibody comprises a heavy chain variable region CDR1 of SEQ ID NO: 7; a heavy chain variable region CDR2 of SEQ ID NO: 8; a heavy chain variable region CDR3 of SEQ ID NO: 10; a light chain variable region CDR1 of SEQ ID NO: 17; a light chain variable region CDR2 of SEQ ID NO: 19; and a light chain variable region CDR3 of SEQ ID NO: 21.

6: The method of claim 3 wherein the antibody comprises a heavy chain variable region as recited in SEQ ID NO: 31 and a light chain variable region as recited in SEQ ID NO: 33.

7: The method of claim 3 wherein the antibody comprises a heavy chain as recited in SEQ ID NO: 41 and a light chain as recited in SEQ ID NO: 39.

8: The method of claim 3 wherein the antibody binds to IL-13 with a K_{D} of 1×10⁻⁹ M or less.

9: The method of claim 3 wherein the antibody is formulated with a pharmaceutically acceptable carrier.

10: The method of claim 3 wherein the antibody is co-administered sequentially or simultaneously with an anti-inflammatory therapeutic agent.

11: A kit comprising a first component and a second component wherein the first component is an anti-IL-13 antibody or pharmaceutical composition comprising an anti-IL-13 antibody and the second component is instructions.

12: The kit of claim 11 further comprising a third component comprising one or more of the following: syringe or other delivery device, adjuvant, or pharmaceutically acceptable formulating solution.


SEQUENCE LISTING (42 sequences)

SEQ ID NO: 1 - protein, 8 aa, Homo sapiens
GFTFSNYG

SEQ ID NO: 2 - protein, 8 aa, Homo sapiens
GFTFSSYG

SEQ ID NO: 3 - protein, 7 aa, Homo sapiens
IWYDGSN

SEQ ID NO: 4 - protein, 8 aa, Homo sapiens
VKGSGDIP

SEQ ID NO: 5 - protein, 9 aa, Homo sapiens
ARLWFGDLD

SEQ ID NO: 6 - protein, 5 aa, Homo sapiens
NYGMH

SEQ ID NO: 7 - protein, 5 aa, Homo sapiens
SYGMH

SEQ ID NO: 8 - protein, 17 aa, Homo sapiens
IIWYDGSNKYYADSVKG

SEQ ID NO: 9 - protein, 9 aa, Homo sapiens
GSGDIPFDY

SEQ ID NO: 10 - protein, 11 aa, Homo sapiens
LWFGDLDAFDI

SEQ ID NO: 11 - protein, 6 aa, Homo sapiens
QSVSSY

SEQ ID NO: 12 - protein, 2 aa, Homo sapiens
DA

SEQ ID NO: 13 - protein, 9 aa, Homo sapiens
HQRSHWPPI

SEQ ID NO: 14 - protein, 9 aa, Homo sapiens
QQRSSWPPV

SEQ ID NO: 15 - protein, 9 aa, Homo sapiens
HQRSSWPPI

SEQ ID NO: 16 - protein, 11 aa, Homo sapiens
RASQSVSSYLA

SEQ ID NO: 17 - protein, 11 aa, Homo sapiens
RAGQSVSSYLV

SEQ ID NO: 18 - skipped

SEQ ID NO: 19 - protein, 7 aa, Homo sapiens
DASNRAT

SEQ ID NO: 20 - protein, 11 aa, Homo sapiens
HQRSHWPPIFT

SEQ ID NO: 21 - protein, 11 aa, Homo sapiens
QQRSSWPPVYT

SEQ ID NO: 22 - protein, 11 aa, Homo sapiens
HQRSSWPPIFT

SEQ ID NO: 23 - protein, 115 aa, Homo sapiens
EVQLVESGGGVVQPGRSLRLSCAASGFTFSNYGMHWVRQAPGKGLEWVAIIWYDGSNKYYADSVKGRFTISRDNSKNTLYLQMNSLRAEDTAVYYCVKGSGDIPFDYWGQGTLVT

SEQ ID NO: 24 - DNA, 345 bp, Homo sapiens
  CDS (1)..(345)
gaagtgcagctggtggagtctgggggaggcgtggtccagcctgggaggtccctgagactctcctgtgcagcgtctggattcaccttcagtaactatggcatgcactgggtccgccaggctccaggcaaggggctggagtgggtggcaattatatggtatgatggaagtaataaatactatgcagactccgtgaagggccgattcaccatctccagagacaattccaagaacacgctgtatctgcaaatgaacagtctgagagccgaggacacggctgtgtattactgtgtgaaaggatctggggatattccctttgactactggggccagggaaccctggtcacc

SEQ ID NO: 25 - protein, 104 aa, Homo sapiens
EIVLTQSPATLSSSPGERATLSCRASQSVSSYLAWYQQKPGQAPRLLIYDASNRATGIPARFSGSGSGTDFTLTISSLEPEDFAVYYCHQRSHWPPIFTFGPGT

SEQ ID NO: 26 - DNA, 312 bp, Homo sapiens
  CDS (1)..(312)
gaaattgtgttgacgcagtctccagccaccctgtcttcgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtctattactgtcatcagcgtagccactggcctcccatattcactttcggccctgggacc

SEQ ID NO: 27 - protein, 115 aa, Homo sapiens; identical to SEQ ID NO: 23

SEQ ID NO: 28 - DNA, 345 bp, Homo sapiens; identical to SEQ ID NO: 24
  CDS (1)..(345)

SEQ ID NO: 29 - protein, 104 aa, Homo sapiens
EIVLTQSPATLSSSPGERATLSCRASQSVSSYLAWYQQKPGQAPRLLIYDASNRATGTPARFSGSGSGTDFTLTISSLEPEDFAVYYCHQRSHWPPIFTFGPGT

SEQ ID NO: 30 - DNA, 312 bp, Homo sapiens
  CDS (1)..(312)
gaaattgtgttgacgcagtccccagccaccctgtcttcgtctccaggggaaagagccaccctctcctgcagggccagtcagagtgttagcagctacttagcctggtaccaacagaaacctggccaggctcccaggctcctcatctatgatgcatccaacagggccactggcaccccagccaggttcagtggcagtgggtctgggacagacttcactctcaccatcagcagcctagagcctgaagattttgcagtctattactgtcatcagcgtagccactggcctcccatattcactttcggccctgggacc

SEQ ID NO: 31 - protein, 117 aa, Homo sapiens
EVQLVESGGGVVQPGRSLRLSCAASGFTFSSYGMHWVRQAPGKGLEWVAIIWYDGSNKYYADSVKGRFTISRDNSKNTLYLQMNSLRAEDTAVYYCARLWFGDLDAFDIWGQGTMVT

SEQ ID NO: 32 - DNA, 351 bp, Homo sapiens
  CDS (1)..(351)
gaagtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcaattatatggtatgatggaagtaataaatactatgcggactccgtgaagggccgattcaccatctccagagacaattccaagaacacgctgtatctgcaaatgaacagcctgagagccgaggacacggctgtgtattactgtgcgaggctatggttcggggacttagatgcttttgatatctggggccaagggacaatggtcacc

SEQ ID NO: 33 - protein, 104 aa, Homo sapiens
EIVLTQSPATLSLSPGERAILSCRAGQSVSSYLVWYQQKPGQAPRLLIYDASNRATGIPARFSGSGSGTDFTLTISSLEPEDFAVYYCQQRSSWPPVYTFGQGT

SEQ ID NO: 34 - DNA, 312 bp, Homo sapiens
  CDS (1)..(312)
gaaattgtgttgacgcagtctccagccaccctgtctttgtctccaggggaaagagccatcctctcctgcagggccggtcagagtgttagcagttacttagtctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgcagcagctggcctccggtgtacacttttggccaggggacc

SEQ ID NO: 35 - protein, 117 aa, Homo sapiens
QVQLVQSGGGVVQPGRSLRLSCAASGFTFSSYGMHWVRQAPGKGLEWVAIIWYDGSNKYYADSVKGRFTISRDNSKNTLYLQMNSLRAEDTAVYYCARLWFGDLDAFDIWGQGTMVT

SEQ ID NO: 36 - DNA, 351 bp, Homo sapiens
  CDS (1)..(351)
caggtgcagctggtgcagtctgggggaggcgtggtccagcctgggaggtccctgagactctcctgtgcggcgtctggattcaccttcagtagctatggcatgcactgggtccgccaggctccaggcaaggggctggagtgggtggcaattatatggtatgatggaagtaataaatactatgcggactccgtgaagggccgattcaccatctccagagacaattccaagaacacgctatatctacaaatgaacagcctgagagccgaggacacggctgtgtattactgtgcgaggctatggttcggggacttagatgcttttgatatctggggccaagggacaatggtcacc

SEQ ID NO: 37 - protein, 104 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCHQRSSWPPIFTFGPGT

SEQ ID NO: 38 - DNA, 312 bp, Homo sapiens
  CDS (1)..(312)
gaaattgtgttgacgcagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcggtttattactgtcatcagcgtagcagctggcccccgatattcactttcggccctgggacc

SEQ ID NO: 39 - protein, 236 aa, Homo sapiens
MSVLTQVLALLLLWLTGTRCEIVLTQSPATLSLSPGERAILSCRAGQSVSSYLVWYQQKPGQAPRLLIYDASNRATGIPARFSGSGSGTDFTLTISSLEPEDFAVYYCQQRSSWPPVYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - DNA, 711 bp, Homo sapiens
  CDS (1)..(711)
atgagtgtgctcactcaggtcctggcgttgctgctgctgtggcttacaggtacgcgttgtgaaattgtgttgacgcagtctccagccaccctgtctttgtctccaggggaaagagccatcctctcctgcagggccggtcagagtgttagcagttacttagtctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgcagcagctggcctccggtgtacacttttggccaggggaccaagcttgaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 41 - protein, 469 aa, Homo sapiens
MAWVWTLPFLMAAAQSVQAEVQLVESGGGVVQPGRSLRLSCAASGFTFSSYGMHWVRQAPGKGLEWVAIIWYDGSNKYYADSVKGRFTISRDNSKNTLYLQMNSLRAEDTAVYYCARLWFGDLDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - DNA, 1410 bp, Homo sapiens
  CDS (1)..(1410)
atggcttgggtgtggaccttgccattcctgatggcagctgcccaaagtgtccaggcagaagtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcaattatatggtatgatggaagtaataaatactatgcggactccgtgaagggccgattcaccatctccagagacaattccaagaacacgctgtatctgcaaatgaacagcctgagagccgaggacacggctgtgtattactgtgcgaggctatggttcggggacttagatgcttttgatatctggggccaagggacaatggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtcgtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga